(19) United States
(12) Patent Application Publication
(10) Pub. No.: US 2015/0272951 A1
(43) Pub. Date: Oct. 1, 2015

(54) COMPOUNDS FOR THE TREATMENT OF LYSOSOMAL STORAGE DISEASES

(71) Applicant: The Hospital for Sick Children, Toronto (CA)

(72) Inventors: Don MAHURAN, Toronto (CA); Michael TROPAK, Toronto (CA); Marco CIUFOLINI, Vancouver (CA)

(21) Appl. No.: 14/728,122

(22) Filed: Jun. 2, 2015

Related U.S. Application Data
(62) Division of application No. 13/024,019, filed on Feb. 9, 2011.
(60) Provisional application No. 61/302,810, filed on Feb. 9, 2010.

(51) Int. Cl. A61K 31/506; A61K 31/505

(52) U.S. Cl. CPC A61K 31/506; A61K 31/505

(57) ABSTRACT

A method of treating a lysosomal storage disease comprises administering a pyrimethamine derivative to a subject in need thereof.

### RELATED APPLICATIONS

This application claims the benefit of U.S. provisional application Ser. No. 61/302,810 filed on Feb. 9, 2010, the disclosure of which is incorporated herein by reference in its entirety.

### FIELD OF THE INVENTION

This invention relates to lysosomal storage diseases. More specifically, this invention is directed to pyrimethamine derivatives and their use in methods of treating lysosomal storage diseases.

### BACKGROUND OF THE INVENTION

Throughout this application, various references are cited in parentheses to describe more fully the state of the art to which this invention pertains. The disclosures of these references are hereby incorporated by reference into the present disclosure in their entirety.

Inter- and intra-cellular macromolecules are disassembled in a stepwise manner in the lysosome and their components are recycled. Many such macromolecules contain carbohydrate moieties. The lysosomal enzymes involved in the turnover of these carbohydrate moieties are specific exoglycosidases, which are synthesized in the endoplasmic reticulum and specifically targeted to the lysosome. If one of these enzymes is deficient, the whole process stops, and partially degraded macromolecules are stored in lysosomes, resulting in the development of various lysosomal storage diseases.

Associated with each lysosomal storage disease is a wide spectrum of clinical phenotypes and/or forms. Generally, a clinical phenotype does not appear unless genetic mutations lead to a >90% reduction in the residual activity of the affected enzyme. Infantile or acute forms of lysosomal storage disease exhibit less than 0.5% residual activity of the affected enzyme and are usually associated with severe neurodegenerative disease and result in death in early infancy. Adult or chronic forms of lysosomal storage disease exhibit approximately 2-5% residual activity of the affected enzyme and may have little or no neurological involvement and may result in a near normal life expectancy. However, even patients with chronic forms of lysosomal storage disease experience a progressive deterioration in their quality-of-life that can ultimately result in institutionalization. The relationship between the amount of residual activity of the affected enzyme and the progression or severity of disease indicates that even very small increases in patients' residual enzyme levels may slow or even reverse the disease process, thereby dramatically enhancing the length and/or quality of their lives (Conzelmann, E., and Sandhoff, K. 1984. Dev. Neurosci. 6:58-71).

Conventional treatments for lysosomal storage diseases include enzyme replacement therapy, bone marrow transplantation, substrate replacement therapy, and enzyme enhancement therapy. Enzyme enhancement therapy has shown promising preclinical results in at least four enzyme deficiencies (Vellodi, A. Loc. Cit; Desnick, R. J., Loc. Cit.) and may be effective in treating neurological forms of lysosomal storage disease.

Enzyme enhancement therapy is achieved using small molecule “chemical chaperones” to stabilize the native conformation of a mutant enzyme in the endoplasmic reticulum (ER), allowing it to escape the ER's quality control system (ERAD) and be transported to the lysosome (Sawkar, A. R., et al. Proc Natl Acad Sci. (USA) 99:15428-15433). To date, many successful chaperones have also been found to be competitive inhibitors or cofactors of their target enzyme and are referred to as pharmacological chaperones. Since, in the case of lysosomal storage diseases, the pharmacological chaperone exerts its activity in the lysosome but not the ER, it has been an important goal to identify pharmacological chaperones that bind more tightly to the target enzyme at the neutral pH of the ER than at the acidic pH of lysosomes. In this way, the pharmacological chaperone may perform its stabilizing function and permit the mutant enzyme to arrive in the lysosome. It is believed that once the pharmacological chaperone-enzyme complex reaches the lysosome, the large amounts of stored substrate(s) will displace the pharmacological chaperone and continue to stabilize the enzyme (Desnick, R. J., Loc. Cit.). Thus, the pharmacological chaperone will cease to inhibit the enzyme and it will be able to carry out its role in the lysosome. Because of the common biochemical features of lysosomal storage diseases, a therapeutic approach that is successful at treating one can usually be adapted for the treatment of others.

Certain lysosomal storage diseases, referred to as GM2 gangliosidoses, are genetic disorders that result from a deficiency of the exoglycosidases that catalyze the biodegradation of fatty acid derivatives known as GM2 gangliosides. One of the key exoglycosidases involved in degradation of GM2 gangliosides is hexosaminidase A (Hex A). Mutations within either of its alpha (encoded by the HEXA gene) or beta subunits (encoded by the HEXB gene) are associated with the development of the GM2 gangliosidoses Tay-Sachs disease or Sandhoff disease, respectively.

U.S. Pat. No. 7,488,721 is directed to compounds with Hex A inhibitory activity for use in the treatment of lysosomal storage diseases such as Tay-Sachs or Sandhoff disease.

Pyrimethamine (PYR) is known to act as an inhibitor of dihydrofolate reductase in Apicomplexans to treat malaria. KSH-10, a derivative of PYR that lacks a chlorine atom, was originally designed as a possible antimalarial compound but was found to have inferior activity against malaria relative to PYR and was not developed further or widely used.

It is now desirable to use PYR derivatives for the treatment of lysosomal storage disorders.

### SUMMARY OF THE INVENTION

The present invention encompasses methods for treating lysosomal storage diseases. In aspects, the method comprises administering a pharmacological chaperone to a subject in need thereof. In an aspect, the pharmacological chaperone is a PYR derivative, such as, for example, KSH-10.

It is now demonstrated that KSH-10 and related PYR derivatives exhibit previously unknown activity in that these PYR derivatives act as Hex A pharmacological chaperones and exhibit lower toxicity than PYR in the treatment of lysosomal storage diseases.

According to an aspect of the present invention, there is provided a method of treating a lysosomal storage disease, the method comprising administering a pyrimethamine derivative to a subject in need thereof. In an aspect, the pyrimethamine derivative is administered in an amount effect to treat, prevent, and/or alleviate the lysosomal storage disease. In another aspect, the pyrimethamine derivative is administered for a time effective to treat, prevent, and/or alleviate the lysosomal storage disease.

According to another aspect, there is provided a use of a pyrimethamine derivative for treating a lysosomal storage disease in a subject in need thereof.

According to another aspect, there is provided a use of a pyrimethamine derivative in the manufacture of a medicament for treating a lysosomal storage disease in a subject in need thereof.

According to an aspect, the lysosomal storage disease is selected from the group consisting of GM1 gangliosidosis, GM2 gangliosidosis, Fabry disease, Gaucher disease, Sanfilippo syndrome, and Morquio disease. In an aspect, the GM2 gangliosidosis is selected from Tay-Sachs disease, Sandhoff disease, and AB variant. In another aspect, the lysosomal storage disease is Tay-Sachs disease.

According to an aspect, the pyrimethamine derivative is of general formula I:

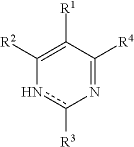

R¹ is a substituted aryl, unsubstituted aryl, substituted heteroaryl, or unsubstituted heteroaryl;
R² is H, NH₂, or alkylamino;
R³ is H, NH₂, ═O, or alkylamino, when R³ is H, NH₂, or alkylamino, [symbol] is a double bond, when R³ is ═O, [symbol] is a double bond; and
R⁴ is a substituted or unsubstituted hydrocarbyl,
wherein when R¹ is 4-chlorophenyl and when [symbol] is a double bond, R² is other than NH₂, R³ is other than NH₂, and R⁴ is other than ethyl.

In an aspect, R¹ is a substituted aryl or unsubstituted heteroaryl; R² is H, NH₂; R³ is NH₂ or alkylamino and [symbol] is a double bond, and R⁴ is a substituted or unsubstituted alkyl.

In another aspect, R¹ is a substituted phenyl, substituted thiophene or unsubstituted thiophene; R² is H, NH₂; R³ is NH₂ or alkylamino and [symbol] is a double bond, and R⁴ is a substituted or unsubstituted C₁-C₁₀ alkyl.

In another aspect, wherein R⁴ is a C₁-C₄ alkyl.

In another aspect, wherein R¹ is:

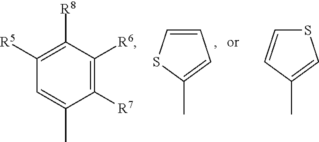

R⁵, R⁶, and R⁷ is independently H, substituted or unsubstituted hydrocarbyl; R⁸ is H, substituted haloalkyl, unsubstituted haloalkyl, halo, substituted hydrocarbyl, unsubstituted hydrocarbyl, substituted alkoxy, or unsubstituted alkoxy.

In a further aspect, wherein R⁵, R⁶, and R⁷ are each independently H or CH₃; and R⁸ is CF₃, CH₃, —O—CH₃, F, H, or Cl.

In an aspect, the pyrimethamine derivative is selected from the group consisting of:

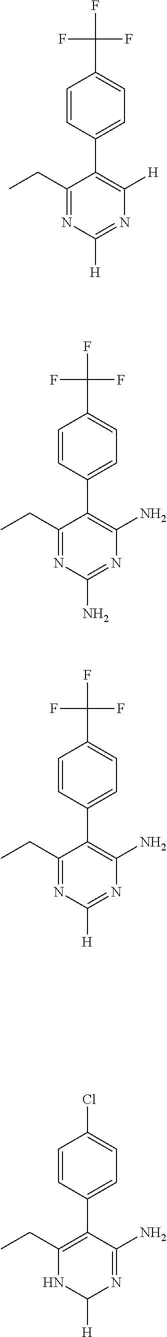 [SMILES: CCc1ncncc1-c1ccc(C(F)(F)F)cc1 ; CCc1nc(N)nc(N)c1-c1ccc(C(F)(F)F)cc1 ; CCc1ncnc(N)c1-c1ccc(C(F)(F)F)cc1 ; CCC1=C(c2ccc(Cl)cc2)C(N)=NCN1]

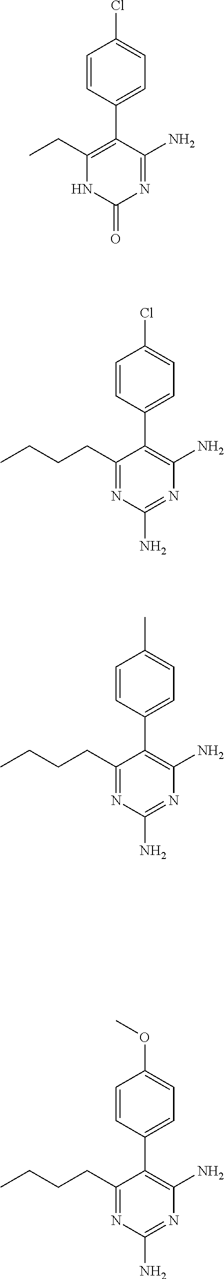 [SMILES: CCc1[nH]c(=O)nc(N)c1-c1ccc(Cl)cc1 ; CCCCc1nc(N)nc(N)c1-c1ccc(Cl)cc1 ; CCCCc1nc(N)nc(N)c1-c1ccc(C)cc1 ; CCCCc1nc(N)nc(N)c1-c1ccc(OC)cc1]

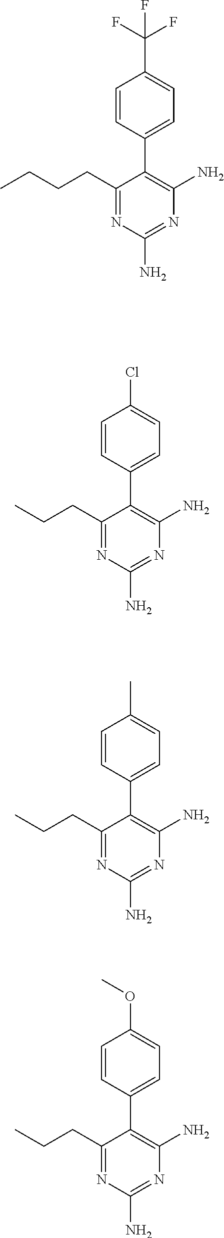 [SMILES: CCCCc1nc(N)nc(N)c1-c1ccc(C(F)(F)F)cc1 ; CCCc1nc(N)nc(N)c1-c1ccc(Cl)cc1 ; CCCc1nc(N)nc(N)c1-c1ccc(C)cc1 ; CCCc1nc(N)nc(N)c1-c1ccc(OC)cc1]

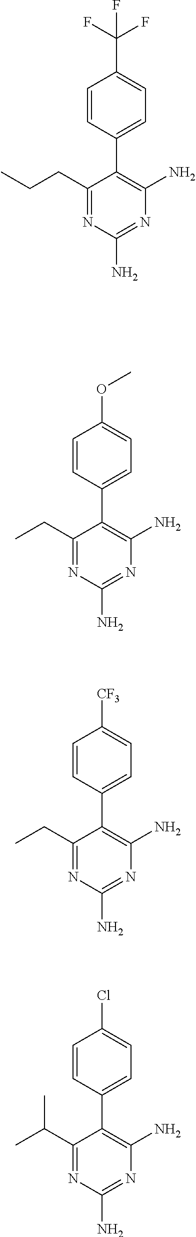 [SMILES: CCCc1nc(N)nc(N)c1-c1ccc(C(F)(F)F)cc1 ; CCc1nc(N)nc(N)c1-c1ccc(OC)cc1 ; CCc1nc(N)nc(N)c1-c1ccc(C(F)(F)F)cc1 ; CC(C)c1nc(N)nc(N)c1-c1ccc(Cl)cc1]

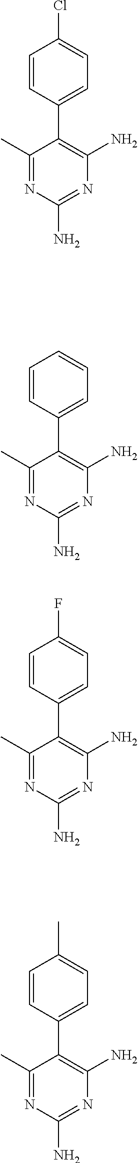 [SMILES: Cc1nc(N)nc(N)c1-c1ccc(Cl)cc1 ; Cc1nc(N)nc(N)c1-c1ccccc1 ; Cc1nc(N)nc(N)c1-c1ccc(F)cc1 ; Cc1ccc(-c2c(C)nc(N)nc2N)cc1]

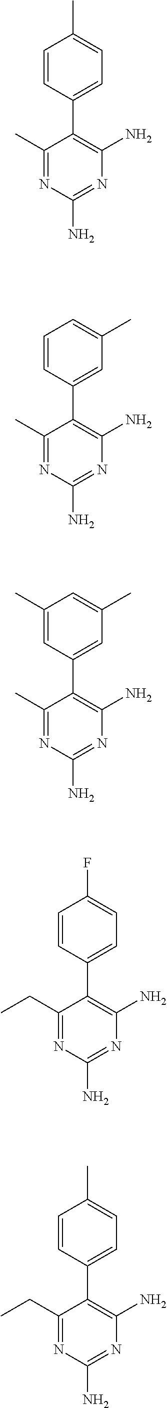

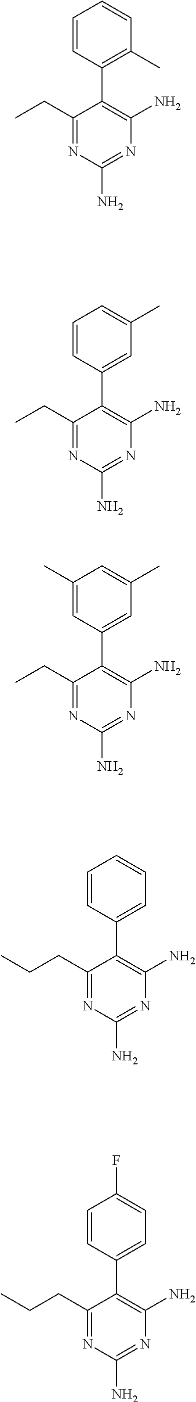

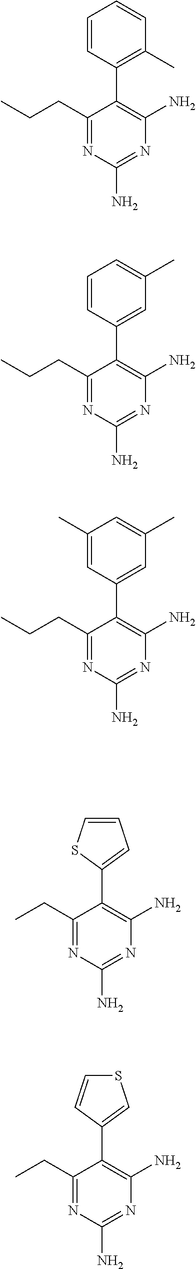

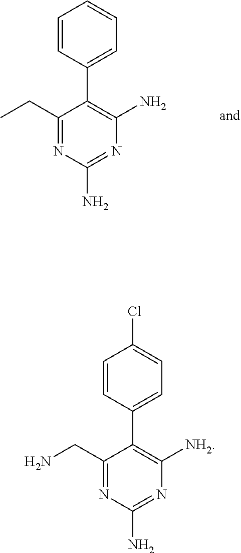 [SMILES: CCc1nc(N)nc(N)c1-c1ccccc1 ; NCc1nc(N)nc(N)c1-c1ccc(Cl)cc1]

In another aspect, the pyrimethamine derivative is

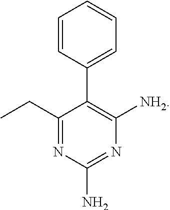 [SMILES: CCc1nc(N)nc(N)c1-c1ccccc1]

According to another aspect, there is provided a pyrimethamine derivative of general formula I:

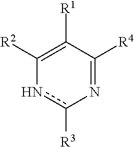

R¹ is a substituted aryl, unsubstituted aryl, substituted heteroaryl, or unsubstituted heteroaryl;
R² is H, NH₂, or alkylamino;
R³ is H, NH₂, ═O, or alkylamino, when R³ is H, NH₂, or alkylamino, [symbol] is a double bond, when R³ is ═O, [symbol] is a double bond; and
R⁴ is a substituted or unsubstituted hydrocarbyl,
wherein when R¹ is phenyl or 4-chlorophenyl and when [symbol] is a double bond, R² is other than NH₂, R³ is other than NH₂, and R⁴ is other than ethyl.

In an aspect, wherein R¹ is a substituted aryl or unsubstituted heteroaryl; R² is H, NH₂; R³ is NH₂ or alkylamino and [symbol] is a double bond, and R⁴ is a substituted or unsubstituted alkyl.

In another aspect, wherein R¹ is a substituted phenyl, substituted thiophene or unsubstituted thiophene; R² is H, NH₂; R³ is NH₂ or alkylamino and [symbol] is a double bond, and R⁴ is a substituted or unsubstituted C₁-C₁₀ alkyl.

In another aspect, wherein R⁴ is a C₁-C₄ alkyl.

In another aspect, R¹ is:

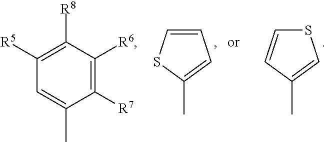

R⁵, R⁶, and R⁷ is independently H, substituted or unsubstituted hydrocarbyl; R⁸ is H, substituted haloalkyl, unsubstituted haloalkyl, halo, substituted hydrocarbyl, unsubstituted hydrocarbyl, substituted alkoxy, or unsubstituted alkoxy.

In another aspect, wherein R⁵, R⁶, and R⁷ are each independently H or CH₃; and R⁸ is CF₃, CH₃, —O—CH₃, F, H, or Cl.

In another aspect, the pyrimethamine derivative is selected from the group consisting of:

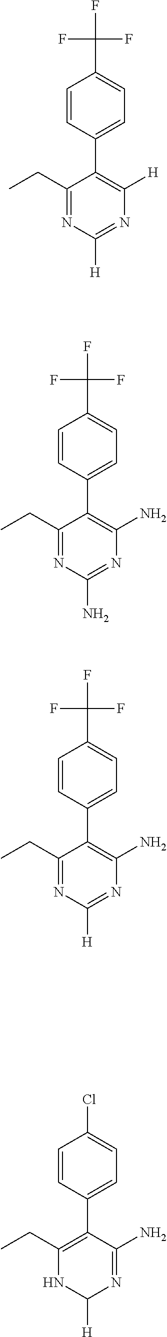 [SMILES: CCc1ncncc1-c1ccc(C(F)(F)F)cc1 ; CCc1nc(N)nc(N)c1-c1ccc(C(F)(F)F)cc1 ; CCc1ncnc(N)c1-c1ccc(C(F)(F)F)cc1 ; CCC1=C(c2ccc(Cl)cc2)C(N)=NCN1]

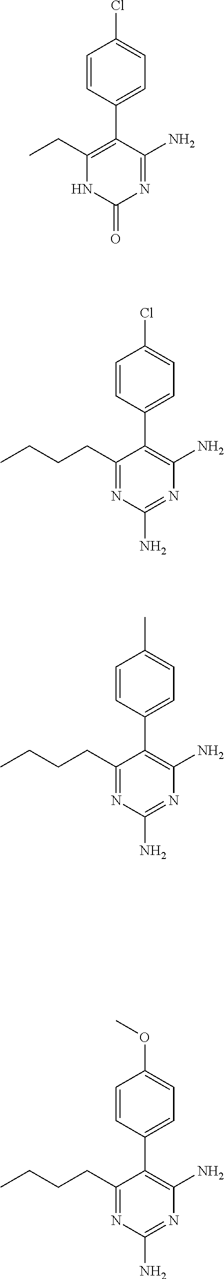 [SMILES: CCc1[nH]c(=O)nc(N)c1-c1ccc(Cl)cc1 ; CCCCc1nc(N)nc(N)c1-c1ccc(Cl)cc1 ; CCCCc1nc(N)nc(N)c1-c1ccc(C)cc1 ; CCCCc1nc(N)nc(N)c1-c1ccc(OC)cc1]

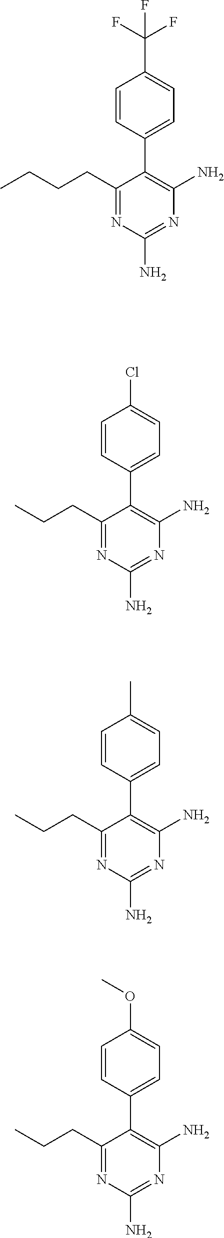 [SMILES: CCCCc1nc(N)nc(N)c1-c1ccc(C(F)(F)F)cc1 ; CCCc1nc(N)nc(N)c1-c1ccc(Cl)cc1 ; CCCc1nc(N)nc(N)c1-c1ccc(C)cc1 ; CCCc1nc(N)nc(N)c1-c1ccc(OC)cc1]

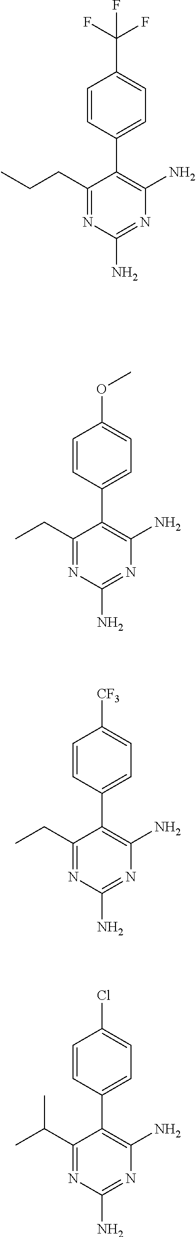 [SMILES: CCCc1nc(N)nc(N)c1-c1ccc(C(F)(F)F)cc1 ; CCc1nc(N)nc(N)c1-c1ccc(OC)cc1 ; CCc1nc(N)nc(N)c1-c1ccc(C(F)(F)F)cc1 ; CC(C)c1nc(N)nc(N)c1-c1ccc(Cl)cc1]

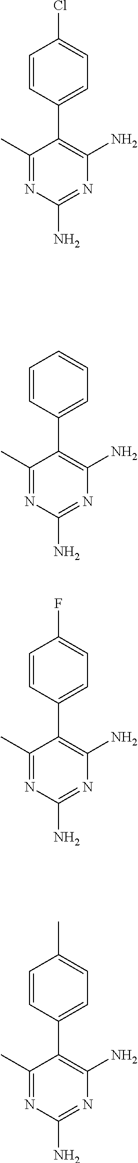 [SMILES: Cc1nc(N)nc(N)c1-c1ccc(Cl)cc1 ; Cc1nc(N)nc(N)c1-c1ccccc1 ; Cc1nc(N)nc(N)c1-c1ccc(F)cc1 ; Cc1ccc(-c2c(C)nc(N)nc2N)cc1]

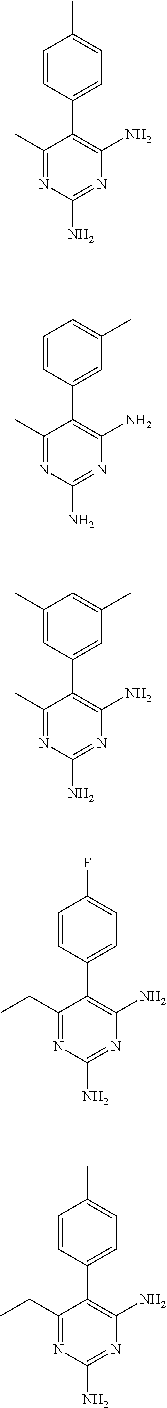

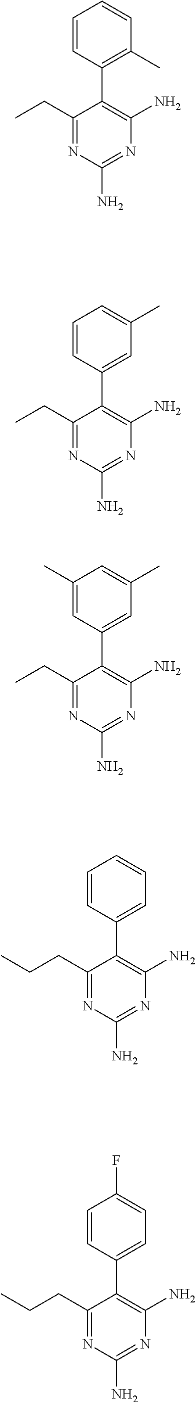

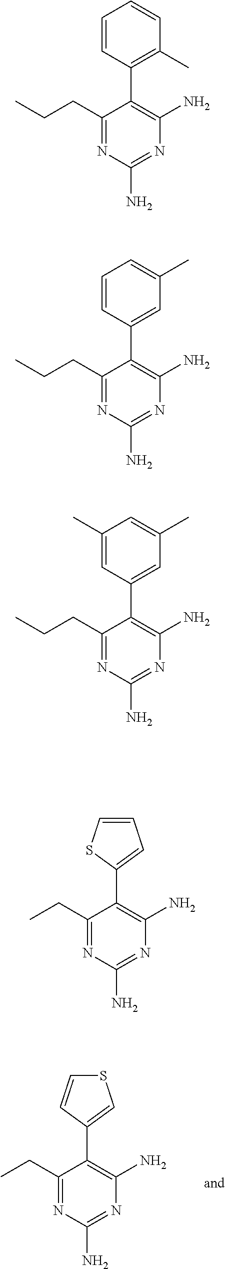

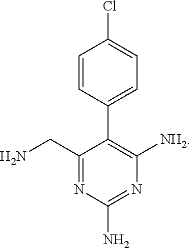 [SMILES: NCc1nc(N)nc(N)c1-c1ccc(Cl)cc1]

In another aspect, the pyrimethamine derivative is for treating a lysosomal storage disease.

In another aspect, the lysosomal storage disease is selected from the group consisting of GM1 gangliosidosis, GM2 gangliosidosis, Fabry disease, Gaucher disease, Sanfilippo syndrome, and Morquio disease. In an aspect, the GM2 gangliosidosis is selected from Tay-Sachs disease, Sandhoff disease, and AB variant. In another aspect, the lysosomal storage disease is Tay-Sachs disease.

Another aspect of the invention are compositions comprising the pyrimethamine derivatives described herein and a pharmaceutically acceptable carrier and/or diluent and/or excipient.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from the detailed description.

### BRIEF DESCRIPTION OF THE DRAWINGS

Embodiments will now be described, by way of example only, with reference to the attached figures, wherein:

FIG. 1 shows the rescue of mutant Hex A activity in fibroblasts expressing the most common mutation (αG269S) associated with late onset GM2 gangliosidosis, adult Tay-Sachs disease (ATSD), by PYR and NGT. ATSD cells were grown in the indicated concentrations of either PYR or NGT for four days. The cells were harvested and assayed for Hex A using a fluorescent artificial substrate, MUGS. The increase in Hex A activity relative to that of untreated cells is given, i.e. no change in activity=1;

FIGS. 2A-2C show the dose response curves for Hex A residual activity following treatment with increasing doses of different PYR derivatives at pH 6.5;

FIGS. 3A-3C show the dose response curves for Hex A residual activity following treatment with increasing doses of different PYR derivatives at pH 4.5;

FIG. 4 shows the IC50 values for Hex A inhibition for PYR derivatives at both pH 4.5 and pH 6.5;

FIG. 5 shows the rescue of mutant Hex A activity in fibroblasts expressing the most common αG269S/G269S mutation associated with late onset GM2 gangliosidosis, ATSD, a rare W474C/Null genotype also associated with ATSD, a rare R504H/R504H mutation associated with late infantile TSD or either of two un-genotyped infantile TSD fibroblast lines by PYR or KSH 3-10. Cells were grown in the indicated concentrations of either drug for five days. The cells were harvested and assayed for Hex A activity using a fluorescent artificial substrate, MUGS. The increase in Hex A activity relative to that of untreated cells is given, i.e. no change in activity=1. The decrease in Hex A activity at high KSH-10 or PYR levels represents cell toxicity;

FIGS. 6A-6B shows the rescue of mutant Hex A activity by PYR derivatives;

FIG. 7 shows the rescue of mutant Hex A activity by the KSH-series of PYR derivatives;

FIG. 8 shows the variation in PYR derivative normalized response parameters;

FIG. 9 shows the correlation between Hex A enhancement and PYR derivative IC50;

FIG. 10 shows the correlation between the calculated IC50 and EC50 values;

FIGS. 11A and 11B show improved enhancement of HexA levels by PYR derivatives (PYRdCl, KSH-10);

FIG. 12 shows increased intracellular GM2 Hydrolysis in PYRdCl (KSH-10); and

FIGS. 13A and 13B show viability of PYR vs PYRdCl treated cells.

### DETAILED DESCRIPTION OF THE INVENTION

The invention provides methods for preventing, inhibiting, alleviating, or treating a lysosomal storage disease. The methods comprise administering a PYR derivative in an amount effective to alleviate or improve a condition, disorder, symptom, or syndrome associated with a lysosomal storage disease. The invention also provides PYR derivatives and their uses alone or in the form of a composition. The methods and compositions of the invention may be used in any type of animal. In an aspect, the animal is a mammal, including a human.

The term “lysosomal storage disease” means any disease resulting from aberrant storage of macromolecules by the lysosome. Lysosomal storage diseases include, but are not limited to, mucopolysaccharidosis diseases, for instance, mucopolysaccharidosis type I, e.g., Hurler syndrome and the variants Scheie syndrome and Hurler-Scheie syndrome (a deficiency in alpha-L-iduronidase); Hunter syndrome (a deficiency of iduronate-2-sulfatase); mucopolysaccharidosis type III, e.g., Sanfilippo syndrome (A, B, C or D; a deficiency of heparan sulfate sulfatase, N-acetyl-alpha-D-glucosaminidase, acetyl CoA:alpha-glucosaminide N-acetyl transferase or N-acetylglucosamine-6-sulfate sulfatase); mucopolysaccharidosis type IV e.g., mucopolysaccharidosis type IV, e.g., Morquio syndrome (a deficiency of galactosamine-6-sulfate sulfatase or beta-galactosidase); mucopolysaccharidosis type VI. e.g., Maroteaux-Lamy syndrome (a deficiency of arylsulfatase B); mucopolysaccharidosis type II; mucopolysaccharidosis type III (A, B, C or D; a deficiency of heparan sulfate sulfatase, N-acetyl-alpha-D-glucosaminidase, acetyl CoA:alpha-glucosaminide N-acetyl transferase or N-acetylglucosamine-6-sulfate sulfatase); mucopolysaccharidosis type IV (A or B; a deficiency of galactosamine-6-sulfatase and beta-galatacosidase); mucopolysaccharidosis type VI (a deficiency of arylsulfatase B); mucopolysaccharidosis type VII (a deficiency in beta-glucuronidase); mucopolysaccharidosis type VIII (a deficiency of glucosamine-6-sulfate sulfatase); mucopolysaccharidosis type IX (a deficiency of hyaluronidase); Tay-Sachs disease (a deficiency in alpha subunit of beta-hexosaminidase); Sandhoff disease (a deficiency in both alpha and beta subunit of beta-hexosaminidase); GM1 gangliosidosis (type I or type II); Fabry disease (a deficiency in alpha galactosidase); metachromatic leukodystrophy (a deficiency of aryl sulfatase A); Pompe disease (a deficiency of acid maltase); fucosidosis (a deficiency of fucosidase); alpha-mannosidosis (a deficiency of alpha-mannosidase); beta-mannosidosis (a deficiency of beta-mannosidase), ceroid lipofuscinosis, and Gaucher disease (types I, II and III; a deficiency in glucocerebrosidase), as well as disorders such as Hermansky-Pudlak syndrome; Amaurotic idiocy; Tangier disease; aspartylglucosaminuria; congenital disorder of glycosylation, type Ia; Chediak-Higashi syndrome; macular dystrophy, corneal, 1; cystinosis, nephropathic; Fanconi-Bickel syndrome; Farber lipogranulomatosis; fibromatosis; geleophysic dysplasia; glycogen storage disease I; glycogen storage disease Ib; glycogen storage disease Ic; glycogen storage disease III; glycogen storage disease IV; glycogen storage disease V; glycogen storage disease VI; glycogen storage disease VII; glycogen storage disease 0; immunoosseous dysplasia, Schimke type; lipidosis; lipase b; mucolipidosis II; mucolipidosis II, including the variant form; mucolipidosis IV; neuraminidase deficiency with beta-galactosidase deficiency; mucolipidosis I; Niemann-Pick disease (a deficiency of sphingomyelinase); Niemann-Pick disease without sphingomyelinase deficiency (a deficiency of a npc1 gene encoding a cholesterol metabolizing enzyme); Refsum disease; Sea-blue histiocyte disease; infantile sialic acid storage disorder; sialuria; multiple sulfatase deficiency; triglyceride storage disease with impaired long-chain fatty acid oxidation; Winchester disease; Wolman disease (a deficiency of cholesterol ester hydrolase); Deoxyribonuclease I-like 1 disorder, arylsulfatase E disorder; ATPase, H+ transporting, lysosomal, subunit 1 disorder; glycogen storage disease IIb; Ras-associated protein rab9 disorder; chondrodysplasia punctata 1, X-linked recessive disorder; glycogen storage disease VIII; lysosome-associated membrane protein 2 disorder; Menkes syndrome; congenital disorder of glycosylation, type Ic; and sialuria.

More specific examples of lysosomal storage diseases include GM1 gangliosidosis, GM2 gangliosidosis, Fabry disease, Gaucher disease, Sanfilippo syndrome, and Morquio syndrome. GM2 gangliosidosis includes Tay-Sachs disease, Sandhoff disease, and an AB variant disease. In an aspect, the lysosomal storage diseases within the scope of the present invention are characterized by having a mutant Hex A protein.

As used herein “PYR derivatives” means derivatives of PYR and do not include PYR. The PYR derivatives may be pharmacological chaperones and both act as inhibitors of its enzyme target and as chemical chaperones, facilitating proper folding of the enzyme target. The enzyme target may be, for example, Hex A or Hex B. The PYR derivatives within the scope of the present invention are derivatives of PYR, which has the following structure:

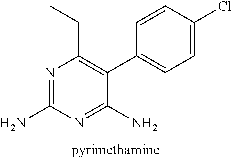 [SMILES: CCc1nc(N)nc(N)c1-c1ccc(Cl)cc1]

The compounds of the present invention may have asymmetric centers, chiral axes, and chiral planes (as described, for example, in: E. L. Eliel and S. H. Wilen, Stereo-chemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present invention. In addition, the compounds disclosed herein may exist as tautomers and both tautomeric forms are intended to be encompassed by the scope of the invention, even though only one tautomeric structure may be depicted.

Generally, reference to a certain element such as hydrogen or H is meant to, if appropriate, include all isotopes of that element.

Where the term “alkyl group” is used, either alone or within other terms such as “haloalkyl group”, it encompasses linear or branched carbon radicals having, for example, one to about ten carbon atoms or, in specific embodiments, one to about four carbon atoms. Examples of such groups include, but are not limited thereto, methyl, ethyl, n-propyl, isopropyl, n-butyl, isobutyl, sec-butyl, tert-butyl, pentyl, iso-amyl, hexyl and the like.

The term “halo” means halogens such as fluorine, chlorine, bromine or iodine atoms.

The term “haloalkyl group” encompasses groups wherein any one or more of the alkyl carbon atoms is substituted with halo as defined above. Specifically encompassed are monohaloalkyl, dihaloalkyl and polyhaloalkyl groups. A monohaloalkyl group, for one example, may have either an iodo, bromo, chloro or fluoro atom within the group. Dihalo and polyhaloalkyl groups may have two or more of the same halo atoms or a combination of different halo groups. “Lower haloalkyl group” encompasses groups having 1-6 carbon atoms. In some embodiments, lower haloalkyl groups have one to three carbon atoms. Examples of haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “alkoxy group” encompasses linear or branched oxy-containing groups each having alkyl portions of, for example and without being limited thereto, one to about ten carbon atoms. In embodiments, alkoxy groups are “lower alkoxy” groups having one to six carbon atoms. Examples of such groups include methoxy, ethoxy, propoxy, butoxy and tert-butoxy. In certain embodiments, lower alkoxy groups have one to three carbon atoms. The “alkoxy” groups may be further substituted with one or more halo atoms, such as fluoro, chloro or bromo, to provide “haloalkoxy” groups. In other embodiments, lower haloalkoxy groups have one to three carbon atoms. Examples of such groups include fluoromethoxy, chloromethoxy, trifluoromethoxy, trifluoroethoxy, fluoroethoxy, and fluoropropoxy.

The term “aromatic group” or “aryl group” means an aromatic group having one or more rings wherein such rings may be attached together in a pendent manner or may be fused. In particular embodiments, an aromatic group is one or two rings. Monocyclic aromatic groups may contain 4 to 10 carbon atoms, typically 4 to 7 carbon atoms, and more typically 6 carbon atoms in the ring. Examples of aromatic groups include, but are not limited to, phenyl, naphthyl, tetrahydronaphthyl, indanyl, biphenyl, phenanthryl, anthryl or acenaphthyl.

The term “heteroatom” means an atom other than carbon. Typically, heteroatoms are selected from the group consisting of sulfur, phosphorus, nitrogen and oxygen atoms. Groups containing more than one heteroatom may contain different heteroatoms.

The term “heteroaromatic group” or “heteroaryl group” means an aromatic group having one or more rings wherein such rings may be attached together in a pendent manner or may be fused, wherein the aromatic group has at least one heteroatom. Monocyclic heteroaromatic groups may contain 4 to 10 member atoms, typically 4 to 7 member atoms, and more typically 5 member atoms in the ring. Examples of heteroaromatic groups include, but are not limited thereto, pyrrole, imidazole, thiazole, oxazole, furan, thiophene, triazole, pyrazole, isoxazole, isothiazole, pyridine, pyrazine, pyridazine, pyrimidine, triazine, indole, benzofuran, benzothiophene, benzimidazole, benzthiazole, quinoline, isoquinoline, quinazoline, quinoxaline and the like.

The term “hydrocarbon group” or “hydrocarbyl group” means a chain of 1 to 25 carbon atoms, typically 1 to 12 carbon atoms, more typically 1 to 10 carbon atoms, and most typically 1 to 8 carbon atoms. Hydrocarbon groups may have a linear or branched chain structure. Typical hydrocarbon groups have one or two branches, typically one branch. Typically, hydrocarbon groups are saturated. Unsaturated hydrocarbon groups may have one or more double bonds, one or more triple bonds, or combinations thereof. Typical unsaturated hydrocarbon groups have one or two double bonds or one triple bond; more typically unsaturated hydrocarbon groups have one double bond.

The term “alkylamino group” denotes amino groups which have been substituted with one alkyl group and with two alkyl groups, including terms “N-alkylamino” and “N,N-dialkylamino”. In embodiments, alkylamino groups are “lower alkylamino” groups having one or two alkyl groups of one to six carbon atoms, attached to a nitrogen atom. In other embodiments, lower alkylamino groups have one to three carbon atoms. Suitable “alkylamino” groups may be mono or dialkylamino such as N-methylamino, N-ethylamino, N,N-dimethylamino, N,N-diethylamino and the like.

The term “suitable substituent”, “substituent” or “substituted” used in conjunction with the groups described herein refers to a chemically and pharmaceutically acceptable group, i.e., a moiety that does not negate the therapeutic activity of the inventive compounds. It is understood that substituents and substitution patterns on the compounds of the invention may be selected by one of ordinary skill in the art to provide compounds that are chemically stable and that can be readily synthesized by techniques known in the art, as well as those methods set forth below. If a substituent is itself substituted with more than one group, it is understood that these multiple groups may be on the same carbon/member atom or on different carbons/member atoms, as long as a stable structure results. Illustrative examples of some suitable substituents include, cycloalkyl, heterocyclyl, hydroxyalkyl, benzyl, carbonyl, halo, haloalkyl, perfluoroalkyl, perfluoroalkoxy, alkyl, alkenyl, alkynyl, hydroxy, oxo, mercapto, alkylthio, alkoxy, aryl or heteroaryl, aryloxy or heteroaryloxy, aralkyl or heteroaralkyl, aralkoxy or heteroaralkoxy, HO—(C═O)—, amido, amino, alkyl- and dialkylamino, cyano, nitro, carbamoyl, alkylcarbonyl, alkoxycarbonyl, alkylaminocarbonyl, dialkylaminocarbonyl, arylcarbonyl, aryloxycarbonyl, alkylsulfonyl, and arylsulfonyl. Typical substituents include aromatic groups, substituted aromatic groups, hydrocarbon groups including alkyl groups such as methyl groups, substituted hydrocarbon groups such as benzyl, and heterogeneous groups including alkoxy groups such as methoxy groups.

The pharmaceutically acceptable salts of the compounds of this invention include the conventional non-toxic salts of the compounds of this invention as formed, e.g., from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

The pharmaceutically acceptable salts of the compounds of this invention can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

The present invention includes pharmaceutically acceptable salts, solvates and prodrugs of the compounds of the invention and mixtures thereof.

The terms “comprising”, “having” and “including”, and various endings thereof, are meant to be open ended, including the indicated component but not excluding other elements.

As the compounds are referred to herein as PYR derivatives, they do not include the parent compound, PYR, itself. In an aspect, the PYR derivative may be of general formula I:

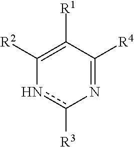

R¹ is a substituted aryl, unsubstituted aryl, substituted heteroaryl, or unsubstituted heteroaryl;
R² is H, NH₂, or alkylamino;
R³ is H, NH₂, ═O, or alkylamino, when R³ is H, NH₂, or alkylamino, [symbol] is a double bond, when R³ is ═O, [symbol] is a double bond; and
R⁴ is a substituted or unsubstituted hydrocarbyl;
wherein when R¹ is phenyl or 4-chlorophenyl and when, [symbol] is a double bond, R² is other than NH₂, R³ is other than NH₂, and R⁴ is other than ethyl. Therefore, the compounds of general formula I exclude PYR and KSH-10.

In specific embodiments, R¹ is a substituted aryl or unsubstituted heteroaryl; R² is H, NH₂; R³ is NH₂ or alkylamino and [symbol] is a double bond, and R⁴ is a substituted or unsubstituted alkyl, such as C₁-C₁₀ alkyl.

In specific embodiments, R¹ is a substituted phenyl, substituted thiophene or unsubstituted thiophene; R² is H, NH₂; R³ is NH₂ or alkylamino and [symbol] is a double bond, and R⁴ is a substituted or unsubstituted alkyl, such as C₁-C₁₀ alkyl.

In other embodiments, R¹ is:

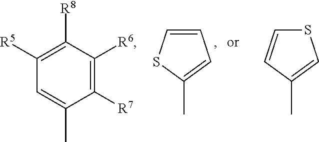

R⁵, R⁶, and R⁷ is independently H, substituted or unsubstituted hydrocarbyl; R⁸ is H, substituted haloalkyl, unsubstituted haloalkyl, halo, substituted hydrocarbyl, unsubstituted hydrocarbyl, substituted alkoxy, or unsubstituted alkoxy. Examples of R⁸, include but are not limited to, CF₃, CH₃, —O—CH₃, F, H, or Cl.

Examples of compounds within the scope of the present invention include the following:

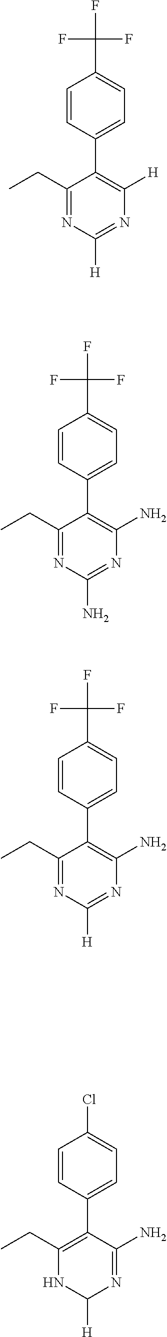 [SMILES: CCc1ncncc1-c1ccc(C(F)(F)F)cc1 ; CCc1nc(N)nc(N)c1-c1ccc(C(F)(F)F)cc1 ; CCc1ncnc(N)c1-c1ccc(C(F)(F)F)cc1 ; CCC1=C(c2ccc(Cl)cc2)C(N)=NCN1]

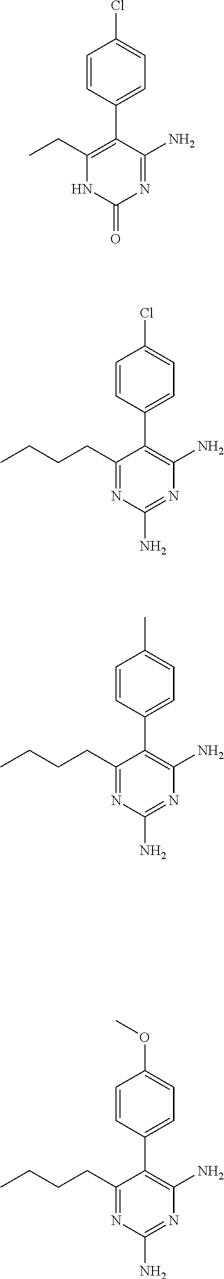 [SMILES: CCc1[nH]c(=O)nc(N)c1-c1ccc(Cl)cc1 ; CCCCc1nc(N)nc(N)c1-c1ccc(Cl)cc1 ; CCCCc1nc(N)nc(N)c1-c1ccc(C)cc1 ; CCCCc1nc(N)nc(N)c1-c1ccc(OC)cc1]

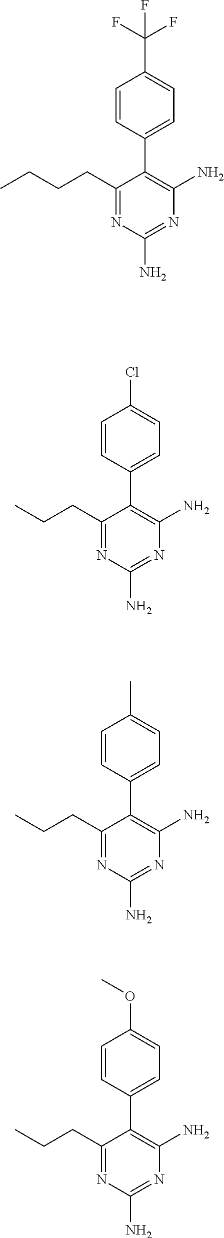 [SMILES: CCCCc1nc(N)nc(N)c1-c1ccc(C(F)(F)F)cc1 ; CCCc1nc(N)nc(N)c1-c1ccc(Cl)cc1 ; CCCc1nc(N)nc(N)c1-c1ccc(C)cc1 ; CCCc1nc(N)nc(N)c1-c1ccc(OC)cc1]

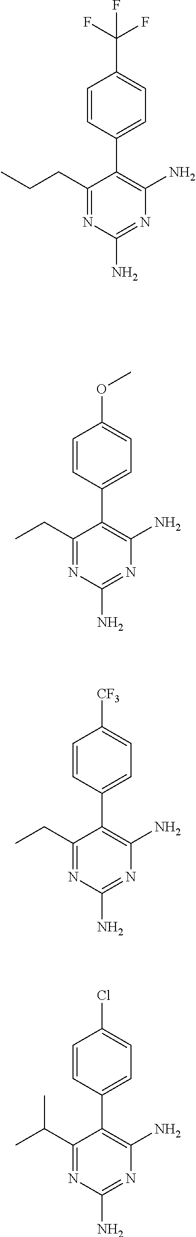 [SMILES: CCCc1nc(N)nc(N)c1-c1ccc(C(F)(F)F)cc1 ; CCc1nc(N)nc(N)c1-c1ccc(OC)cc1 ; CCc1nc(N)nc(N)c1-c1ccc(C(F)(F)F)cc1 ; CC(C)c1nc(N)nc(N)c1-c1ccc(Cl)cc1]

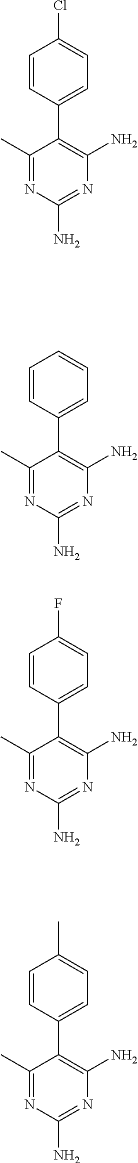 [SMILES: Cc1nc(N)nc(N)c1-c1ccc(Cl)cc1 ; Cc1nc(N)nc(N)c1-c1ccccc1 ; Cc1nc(N)nc(N)c1-c1ccc(F)cc1 ; Cc1ccc(-c2c(C)nc(N)nc2N)cc1]

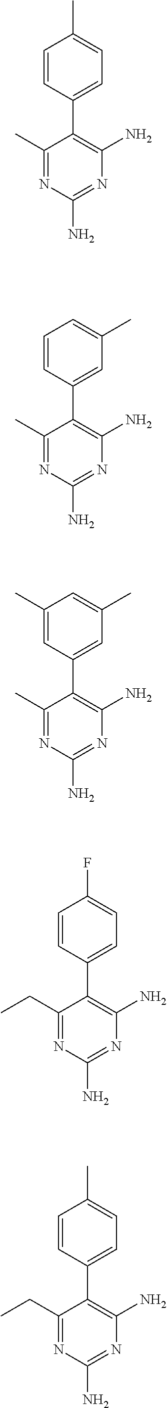

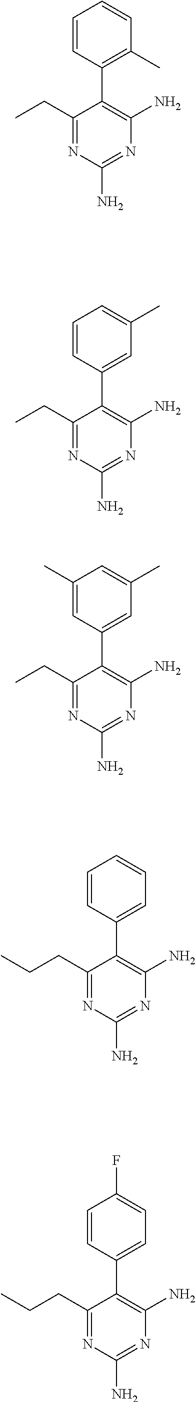

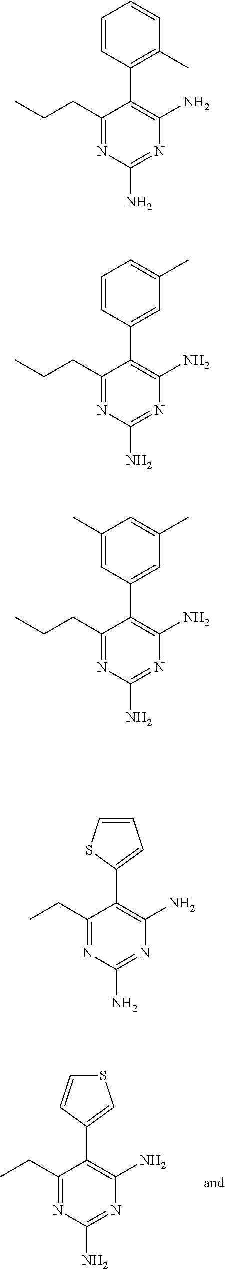

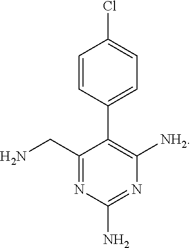 [SMILES: NCc1nc(N)nc(N)c1-c1ccc(Cl)cc1]

In addition to the PYR derivatives described herein, compound:

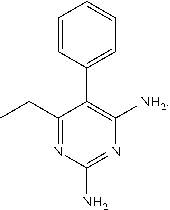 [SMILES: CCc1nc(N)nc(N)c1-c1ccccc1]

which is referred to as “KSH-10” or “KSH3-10”, can be administered in an amount effective to alleviate or improve a condition, disorder, symptom, or syndrome associated with a lysosomal storage disease. A composition may also be used.

All stereoisomers are included within the scope of the invention, on their own as pure compounds as well as mixtures thereof. Unless otherwise indicated, individual enantiomers, diastereomers, geometrical isomers, and combinations and mixtures thereof are all encompassed by the present invention. Polymorphic crystalline forms and solvates are also encompassed within the scope of this invention.

The present invention includes within its scope prodrugs of the compounds of this invention. Such prodrugs are in general functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to a subject in need thereof. Conventional procedures for the selection and preparation of suitable prodrug derivatives are described, for example, in Wermuth, “Designing Prodrugs and Bioprecursors,” in Wermuth, ed., The Practice of Medicinal Chemistry, 2nd Ed., pp. 561-586 (Academic Press 2003), the disclosure of which is incorporated herein by reference. Prodrugs include esters that hydrolyze in vivo (for example in the human body) to produce a compound of this invention or a salt thereof. Suitable ester groups include, without limitation, those derived from pharmaceutically acceptable aliphatic carboxylic acids, particularly zalkanoic, alkenoic, cycloalkanoic and alkanedioic acids, in which each alkyl or alkenyl moiety preferably has no more than six carbon atoms. Illustrative esters include but are not limited to formates, acetates, propionates, butyrates, acrylates, citrates, succinates, and ethylsuccinates.

The PYR derivatives may be provided in a purified and isolated form, for example following column chromatography, high-pressure liquid chromatography, recrystallization, or other purification technique.

The PYR derivatives may be used in a pharmaceutical formulation or composition comprising a compound of this invention and an excipient. Excipients that may be used include, for example, carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003), the disclosure of which is incorporated herein by reference.

The composition may be in any suitable form such as solid, semisolid, or liquid form. In general, the pharmaceutical preparation will contain one or more of the compounds of the invention as an active ingredient in admixture with an organic or inorganic carrier or excipient suitable for external, enteral, or parenteral application. The active ingredient may be compounded, for example, with the usual non-toxic, pharmaceutically acceptable carriers for tablets, pellets, capsules, suppositories, pessaries, solutions, emulsions, suspensions, and any other form suitable for use. The carriers that can be used include, for example, water, glucose, lactose, gum acacia, gelatin, mannitol, starch paste, magnesium trisilicate, talc, corn starch, keratin, colloidal silica, potato starch, urea, and other carriers suitable for use in manufacturing preparations, in solid, semi-solid, or liquified form. In addition, auxiliary stabilizing, thickening, and coloring agents and perfumes may be used.

Where applicable, compounds of this invention may be formulated as microcapsules and nanoparticles. General protocols are described for example, in Bosch et al., U.S. Pat. No. 5,510,118 (1996); De Castro, U.S. Pat. No. 5,534,270 (1996); and Bagchi et al., U.S. Pat. No. 5,662,883 (1997), which are all incorporated herein by reference. By increasing the ratio of surface area to volume, these formulations allow for the oral delivery of compounds that would not otherwise be amenable to oral delivery.

Dosage levels of the compounds of the present invention may be of the order from about 0.001 mg to about 10000 mg per kilogram of body weight per day, from 0.1 mg to about 100 mg per kilogram of body weight per day, or from about 1 mg to about 50 mg per kilogram of body weight per day. The dosage levels may be from about 0.5 mg to about 2000 mg per kilogram of body weight per day, corresponding to 35 mg to 14000 mg per patient per day, assuming a 70 kg patient. The compounds of the present invention may be administered once or on an intermittent basis, such as, for example, at hourly, daily, semi-weekly, weekly, semi-monthly, or monthly intervals.

The amount of active ingredient that may be combined with the carrier materials to produce a single dosage form will vary depending upon the host treated and the particular mode of administration. For example, a formulation intended for oral administration to humans may contain carrier material, which may vary from about 5 percent to about 95 percent of the total composition. Dosage unit forms may contain from about 5 mg to about 500 mg of active ingredient.

It will be understood, however, that the specific dose level for any particular patient will depend on a variety of factors. These factors include the activity of the specific compound employed; the age, body weight, general health, sex, and diet of the subject; the time and route of administration and the rate of excretion of the drug; whether a drug combination is employed in the treatment; and the severity of the particular disease or condition for which therapy is sought.

One or more suitable unit dosage forms comprising the PYR derivatives of the invention may be administered by a variety of routes including oral, or parenteral, including by rectal, buccal, vaginal and sublingual, transdermal, subcutaneous, intravenous, intramuscular, intraperitoneal, intrathoracic, intrapulmonary and intranasal routes. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

Additionally, the PYR derivatives may be formulated as sustained release dosage forms and the like. The formulations can be so constituted that they release the active ingredient only or preferably in a particular part of the intestinal or respiratory tract, possibly over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, catheters, peritoneal dialysis tubing, and the like.

The PYR derivatives of the invention may be delivered via patches for transdermal administration. See U.S. Pat. No. 5,560,922, which is incorporated herein by reference, for examples of patches suitable for transdermal delivery of a therapeutic agent. Patches for transdermal delivery may comprise a backing layer and a polymer matrix which has dispersed or dissolved therein a PYR derivative, along with one or more skin permeation enhancers. The backing layer may be made of any suitable material which is impermeable to the therapeutic agent. The backing layer serves as a protective cover for the matrix layer and provides also a support function. The backing may be formed so that it is essentially the same size layer as the polymer matrix or it may be of larger dimension so that it can extend beyond the side of the polymer matrix or overlay the side or sides of the polymer matrix and then can extend outwardly in a manner that the surface of the extension of the backing layer can be the base for an adhesive means Alternatively, the polymer matrix may contain, or be formulated of, an adhesive polymer, such as polyacrylate or acrylate/vinyl acetate copolymer. For long-term applications it might be desirable to use microporous and/or breathable backing laminates, so hydration or maceration of the skin can be minimized.

For administration to the upper (nasal) or lower respiratory tract by inhalation, the PYR derivatives of the invention may be conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the PYR derivative and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the PYR derivative may be administered via nose drops, a liquid spray, such as via a plastic bottle atomizer or metered-dose inhaler. Typical of atomizers are the Mistometer (Wintrop) and the Medihaler (Riker).

The local delivery of the PYR derivatives of the invention may also be by a variety of techniques that administer the derivative at or near the site of disease. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

For topical administration, the PYR derivatives may be formulated as is known in the art for direct application to a target area. Conventional forms for this purpose include wound dressings, coated bandages or other polymer coverings, ointments, creams, lotions, pastes, jellies, sprays, and aerosols, as well as in toothpaste and mouthwash, or by other suitable forms, e.g., via a coated condom. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredients can also be delivered via iontophoresis, e.g., as disclosed in U.S. Pat. Nos. 4,140,122; 4,383,529; or 4,051,842, which are incorporated herein by reference. The percent by weight of a PYR derivative of the invention present in a topical formulation will depend on various factors, but generally will be from 0.01% to 95% of the total weight of the formulation, and typically 0.1-25% by weight.

When desired, the above-described formulations may be adapted to give sustained release of the active ingredient employed, e.g., by combination with certain hydrophilic polymer matrices, e.g., comprising natural gels, synthetic polymer gels or mixtures thereof.

Drops, such as eye drops or nose drops, may be formulated with an aqueous or non-aqueous base also comprising one or more dispersing agents, solubilizing agents or suspending agents. Liquid sprays are conveniently delivered from pressurized packs. Drops may be delivered via a simple eye dropper-capped bottle, or via a plastic bottle adapted to deliver liquid contents dropwise, via a specially shaped closure.

The PYR derivatives may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; mouthwashes comprising the composition of the present invention in a suitable liquid carrier; and pastes and gels, e.g., toothpastes or gels, comprising the composition of the invention.

The formulations and compositions described herein may also contain other ingredients such as antimicrobial agents, or preservatives. Furthermore, the active ingredients may also be used in combination with other therapeutic agents. In particular, the PYR derivatives may be administered alone or in combination with other PYR derivatives or with other conventional treatments for lysosomal storage diseases.

Although each of the derivatives described herein have different specific structures, they each act as pharmacological chaperones and thus have utility in the treatment of lysosomal storage diseases.

### EXAMPLES

### Example 1

### Identification and Characterization of PYR as a Hex A Inhibitor

The NINDS library was screened for compounds having Hex A inhibitory activity. PYR (IC50˜8 μM at pH 4.5) and thioguanine (IC50˜2 mM) were identified as candidate inhibitors. The pK_{HA} of PYR was compared to NAG-thiazoline (NGT), a known Hex A inhibitor. Whereas NGT has pK_{HA} of 4.5, PYR has a pK_{HA} of 6.5. This difference in pK_{HA} indicates that different amino acid side chains in or near the active site of Hex A are involved in binding NGT versus PYR.

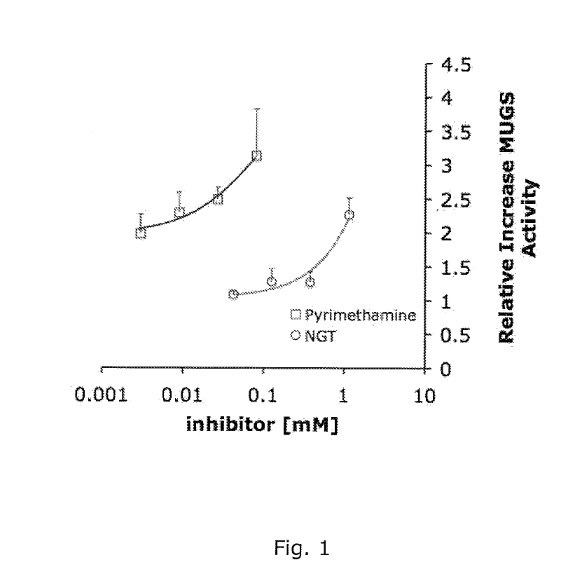

Moreover, PYR was found to exhibit an IC50 of 3.4 μg/mL at pH 6.5 and an IC50 of 13.7 μg/mL at pH 4.5. This inhibitory profile of PYR indicates that it should be a better pharmacological chaperone than NGT for treating chronic GM2 gangliosidosis as it will bind tighter to Hex A in the neutral ER than in the acidic lysosome. Indeed, PYR was better able to rescue mutant Hex A activity in fibroblasts expressing a common mutation (αG269S) associated with adult Tays-Sachs disease (FIG. 1). Additionally, PYR is expected to have a better bio-availability than NGT.

### Example 2

### Preparation of PYR Derivatives

Using a Selective Optimization Of Side Activities (SOSA) approach, several PYR derivatives were conceived and prepared. The general scheme for the synthesis of PYR and its analogs is as follows:

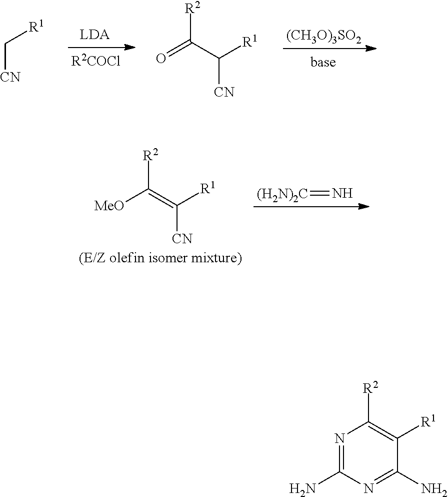

For example, the derivative KSH3-10 was prepared as follows:

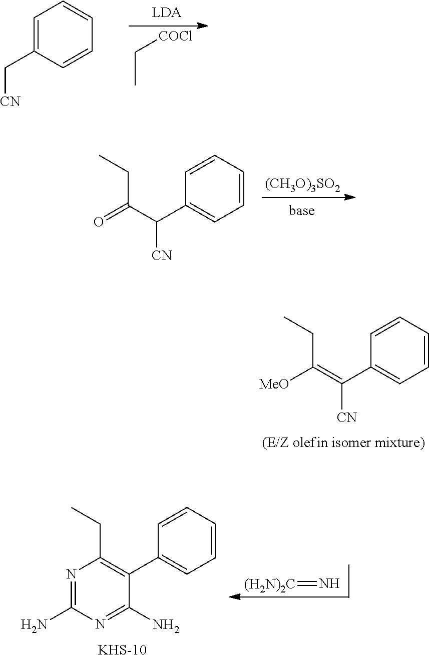

Other prepared derivatives include the structures shown in Table 1.

TABLE 1
Structures, names, molecular weights and concentrations
of PYR derivatives.

| | | Molecular | Stock |
|---|---|---|---|
| | | Weight | Conc. |
| Structure | Name | (g/mol) | (mg/ml) |
| 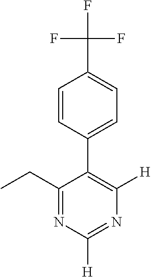 [SMILES: CCc1ncncc1-c1ccc(C(F)(F)F)cc1] | vsa4 | 252 | 26.67 |
| 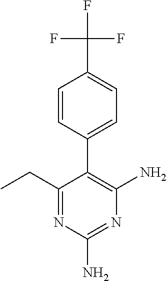 [SMILES: CCc1nc(N)nc(N)c1-c1ccc(C(F)(F)F)cc1] | vsa5 | 282 | 8.6 |
| 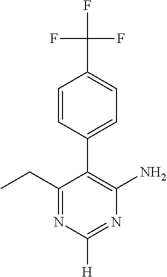 [SMILES: CCc1ncnc(N)c1-c1ccc(C(F)(F)F)cc1] | vsa6 | 267 | 5 |
| 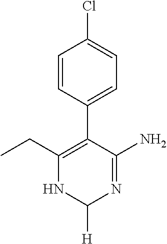 [SMILES: CCC1=C(c2ccc(Cl)cc2)C(N)=NCN1] | vsa8 | 234 | 9.8 |
| 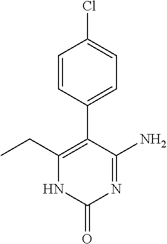 [SMILES: CCc1[nH]c(=O)nc(N)c1-c1ccc(Cl)cc1] | vsa9 | 250 | 5 |
| 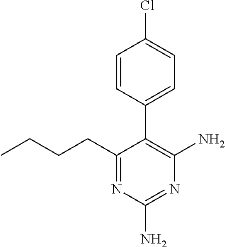 [SMILES: CCCCc1nc(N)nc(N)c1-c1ccc(Cl)cc1] | zjm1-91 | 296 | 10 |
| 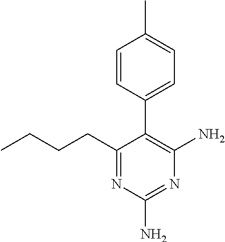 [SMILES: CCCCc1nc(N)nc(N)c1-c1ccc(C)cc1] | zjm1-111 | 256 | 10 |
| 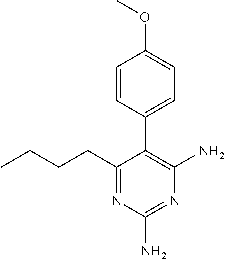 [SMILES: CCCCc1nc(N)nc(N)c1-c1ccc(OC)cc1] | zjm1-113 | 272 | 10 |
| 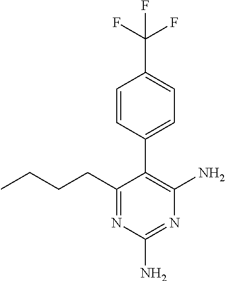 [SMILES: CCCCc1nc(N)nc(N)c1-c1ccc(C(F)(F)F)cc1] | zjm1-115 | 1116 | 10 |
| 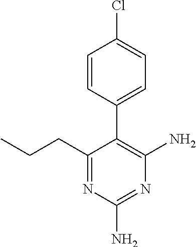 [SMILES: CCCc1nc(N)nc(N)c1-c1ccc(Cl)cc1] | zjm1-135 | 263 | 10 |
| 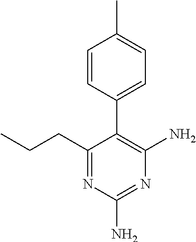 [SMILES: CCCc1nc(N)nc(N)c1-c1ccc(C)cc1] | zjm1-137 | 242 | 10 |
| 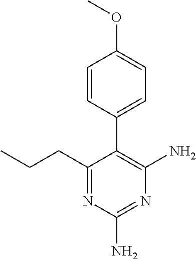 [SMILES: CCCc1nc(N)nc(N)c1-c1ccc(OC)cc1] | zjm1-139 | 258 | 10 |
| 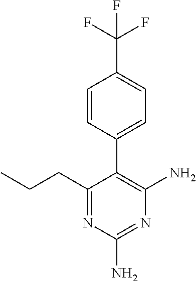 [SMILES: CCCc1nc(N)nc(N)c1-c1ccc(C(F)(F)F)cc1] | zjm1-141 | 296 | 10 |
| 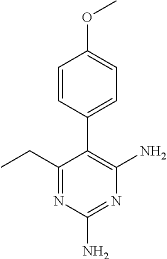 [SMILES: CCc1nc(N)nc(N)c1-c1ccc(OC)cc1] | jts14 | 249 | 3.333 |
| 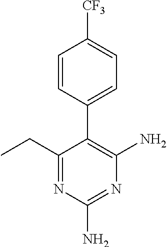 [SMILES: CCc1nc(N)nc(N)c1-c1ccc(C(F)(F)F)cc1] | jts16 | 244 | 10 |
| 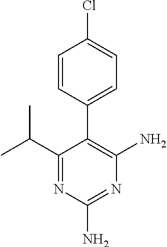 [SMILES: CC(C)c1nc(N)nc(N)c1-c1ccc(Cl)cc1] | jts20 | 263 | 10 |
| 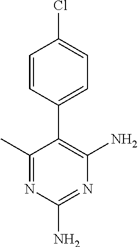 [SMILES: Cc1nc(N)nc(N)c1-c1ccc(Cl)cc1] | jts22 | 235 | 10 |
| 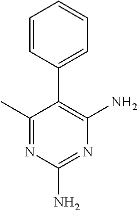 | ksh3-5 | 200 | 10 |
| 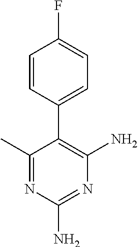 [SMILES: Cc1nc(N)nc(N)c1-c1ccc(F)cc1] | ksh3-14a | 218 | 10 |
| 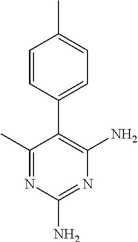 [SMILES: Cc1ccc(-c2c(C)nc(N)nc2N)cc1] | ksh3-14b | 214 | 10 |
| 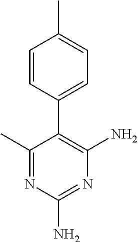 [SMILES: Cc1ccc(-c2c(C)nc(N)nc2N)cc1] | ksh3-14c | 214 | 10 |
| 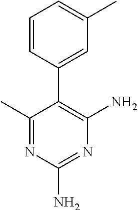 | ksh3-14d | 214 | 10 |
| 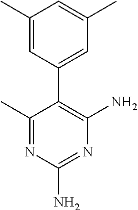 | ksh3-14e | 228 | 10 |
| 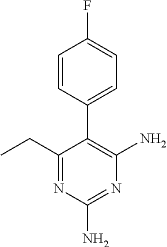 [SMILES: CCc1nc(N)nc(N)c1-c1ccc(F)cc1] | ksh3-17 | 246 | 10 |
| 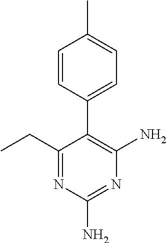 [SMILES: CCc1nc(N)nc(N)c1-c1ccc(C)cc1] | ksh3-19b | 228 | 10 |
| 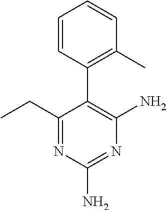 [SMILES: CCc1nc(N)nc(N)c1-c1ccccc1C] | ksh3-19c | 228 | 10 |
| 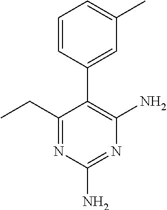 [SMILES: CCc1nc(N)nc(N)c1-c1cccc(C)c1] | ksh3-19d | 228 | 10 |
| 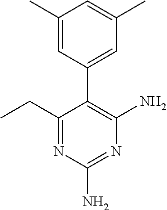 [SMILES: CCc1nc(N)nc(N)c1-c1cc(C)cc(C)c1] | ksh3-19e | 242 | 10 |
| 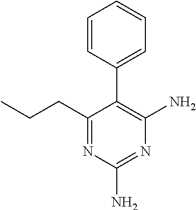 [SMILES: CCCc1nc(N)nc(N)c1-c1ccccc1] | ksh3-29 | 228 | 10 |
| 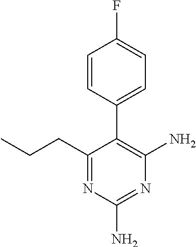 [SMILES: CCCc1nc(N)nc(N)c1-c1ccc(F)cc1] | ksh3-33a | 246 | 10 |
| 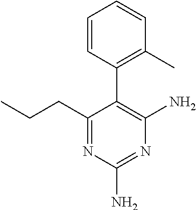 [SMILES: CCCc1nc(N)nc(N)c1-c1ccccc1C] | ksh3-33c | 242 | 10 |
| 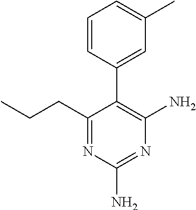 [SMILES: CCCc1nc(N)nc(N)c1-c1cccc(C)c1] | ksh3.33d | 242 | 10 |
| 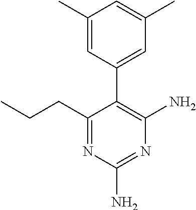 [SMILES: CCCc1nc(N)nc(N)c1-c1cc(C)cc(C)c1] | ksh3.33e | 256 | 10 |
| 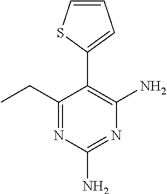 | zjm7-67 | 220 | 10 |
| 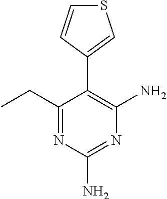 | zjm7-69 | 220 | 10 |
| 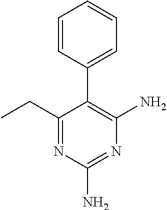 [SMILES: CCc1nc(N)nc(N)c1-c1ccccc1] | ksh3-10 | 214 | 10 |
| 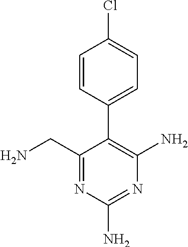 [SMILES: NCc1nc(N)nc(N)c1-c1ccc(Cl)cc1] | zjm7-27 | 250 | 10 |

### Example 3

### IC50 Values at Neutral and Acidic pH

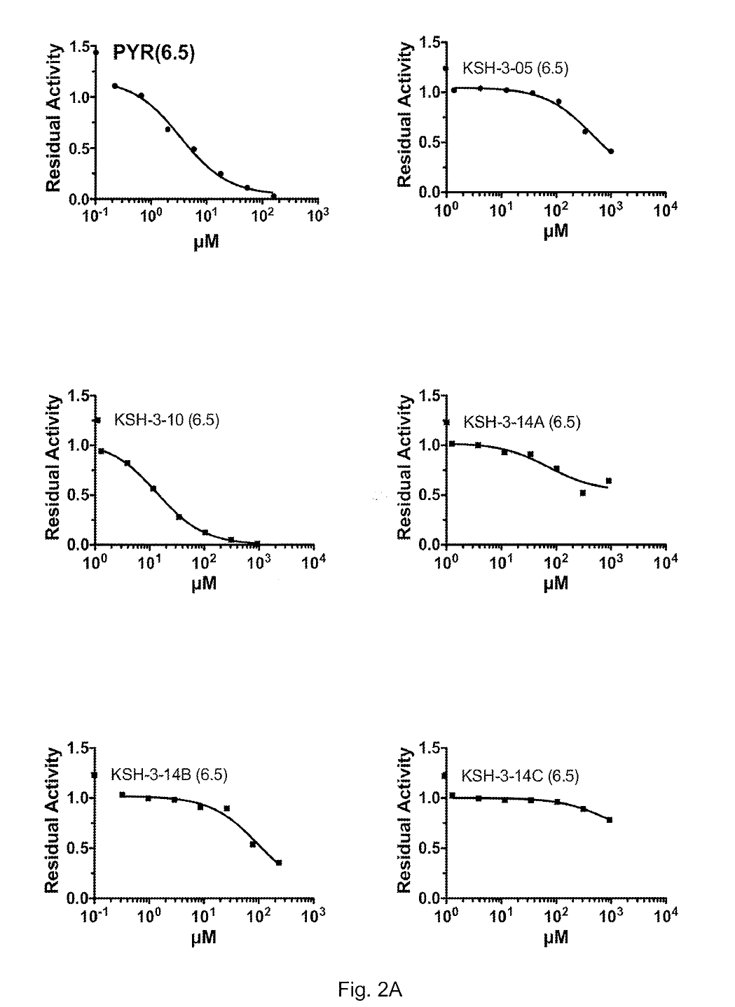

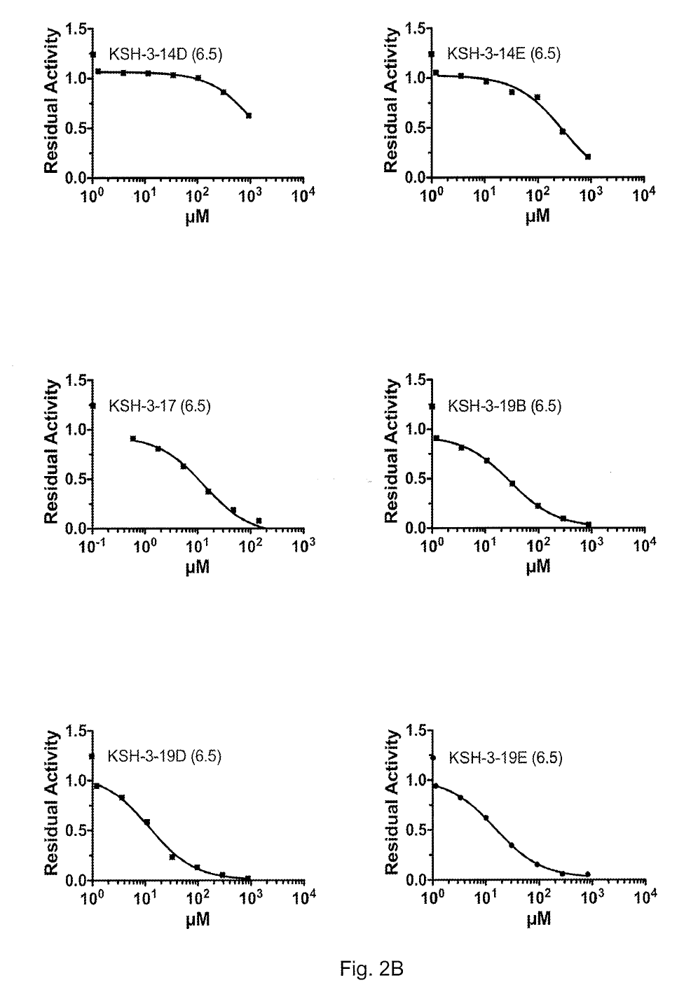

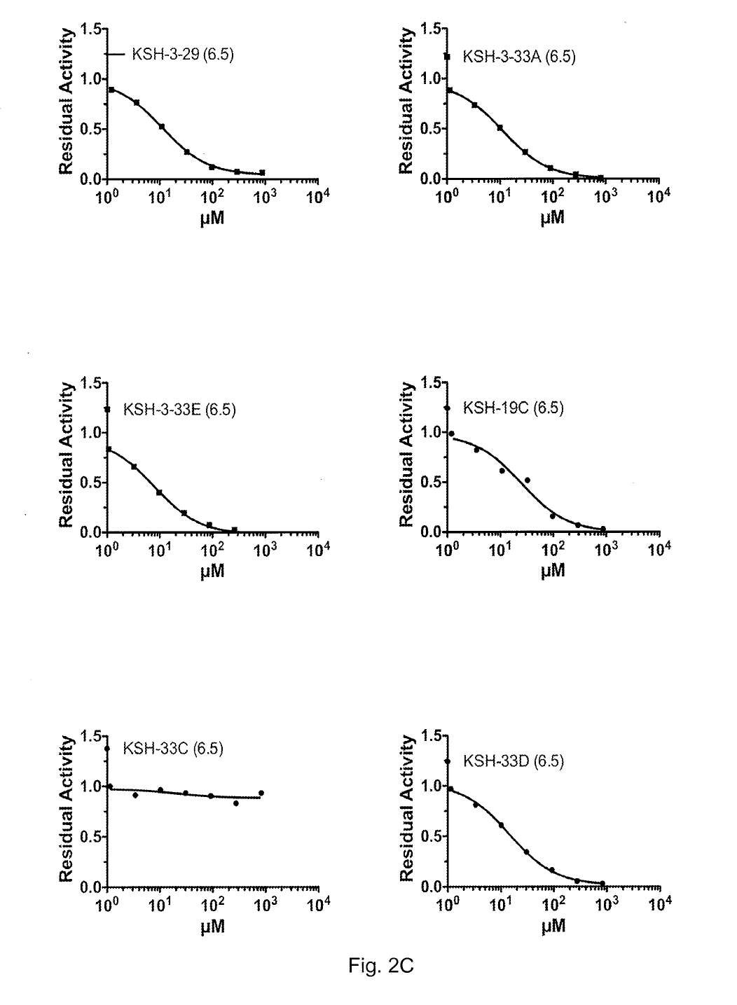

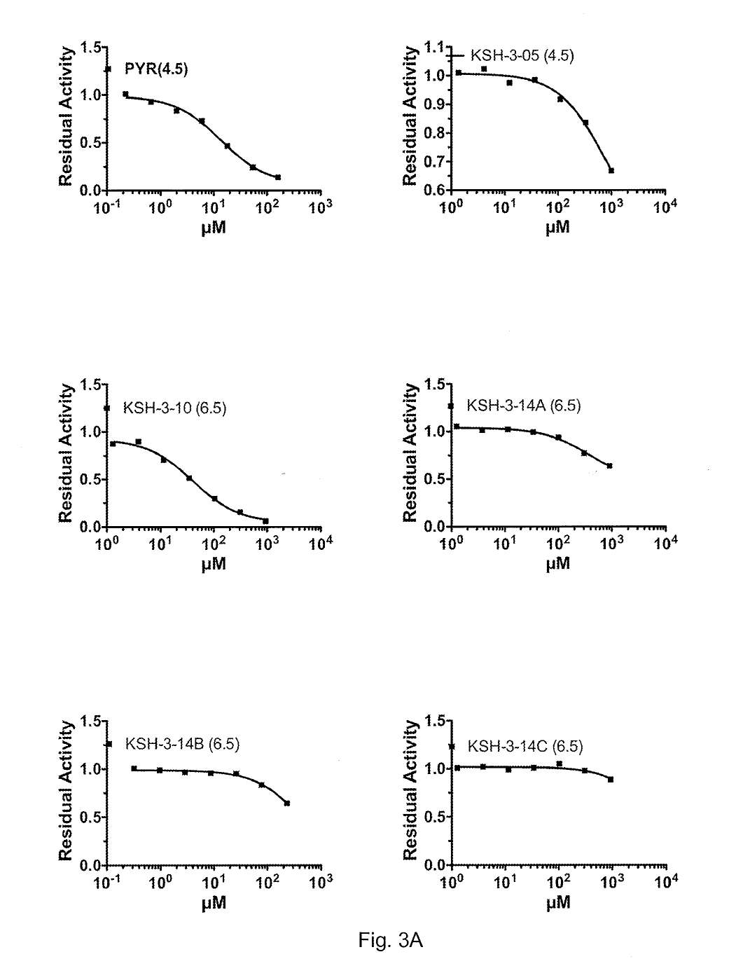

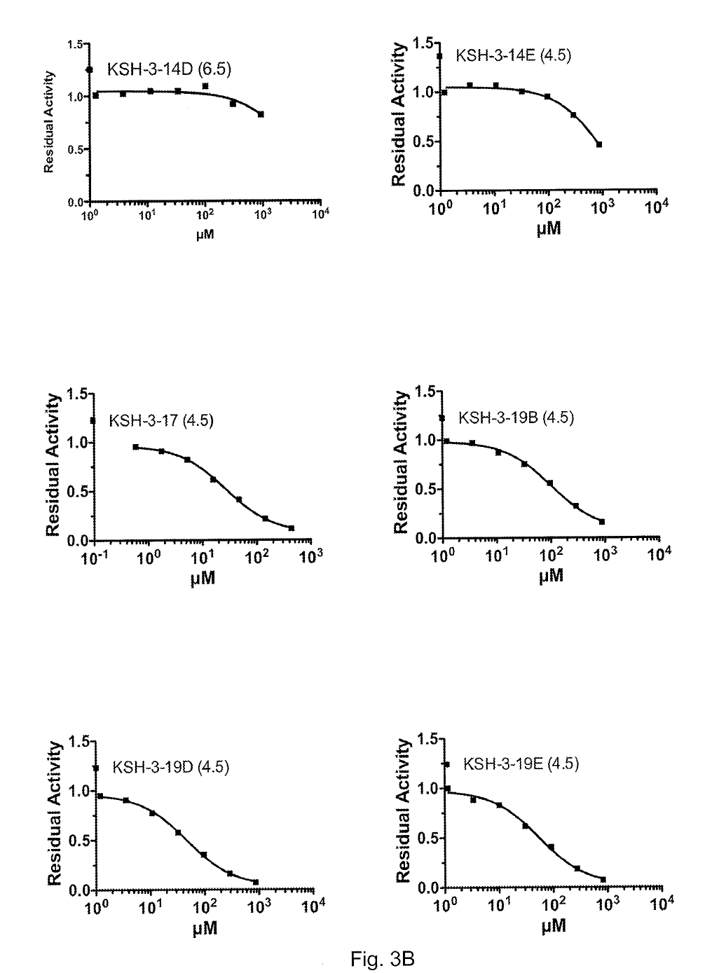

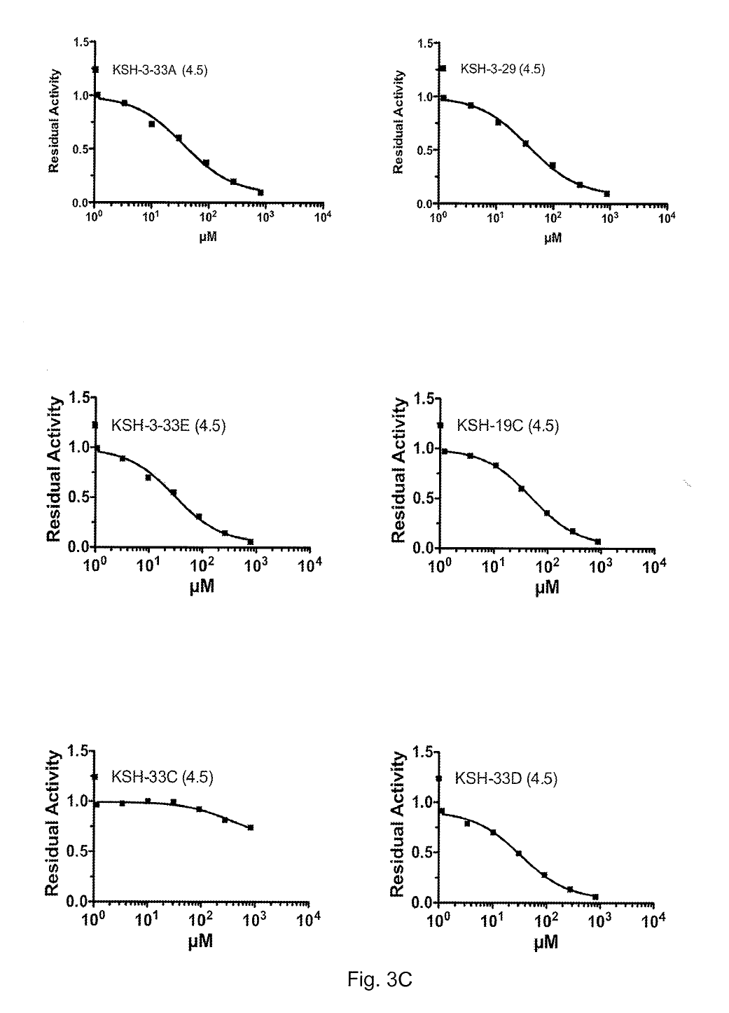

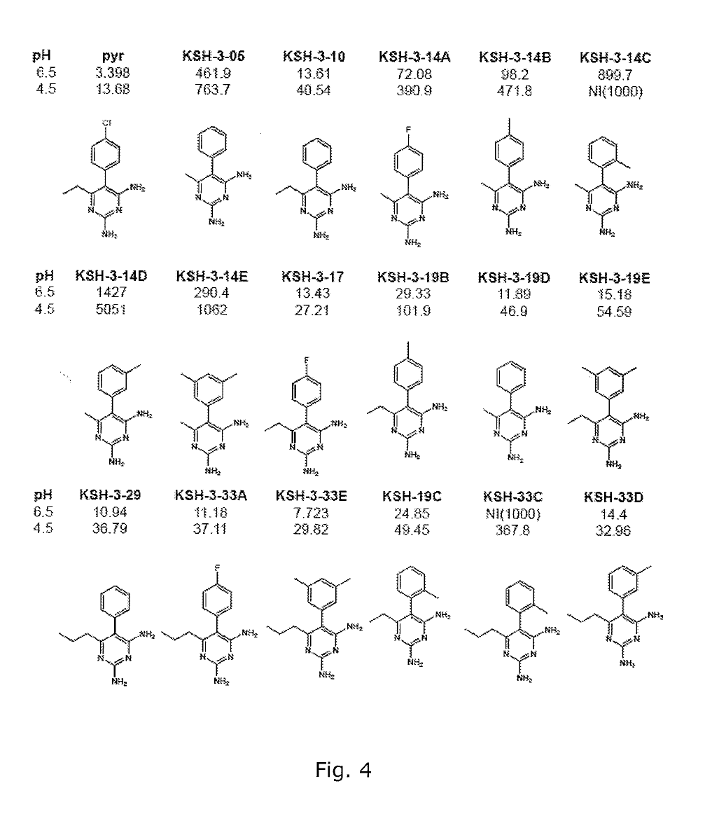

PYR, KSH-10 and the other KSH-series derivatives were tested to determine their IC50 values for Hex A inhibition at both pH 4.5 and pH 6.5. The results are shown in FIGS. 2-4. FIGS. 2A and 2B show the dose response curves for Hex A residual activity following treatment with increasing doses of the respective derivative at pH 6.5. FIG. 2C shows all of these dose response curves together on a single graph. FIGS. 3A-C show similar dose response curves carried out at pH 4.5. FIG. 4 shows the actual IC50 values that were determined from these dose response curves.

As can be seen from these figures, with the exception of KSH-33C, all of the derivatives demonstrated an IC50 value that was lower at pH 6.5 than it was at pH 4.5. This indicates that all of these derivatives would be good candidates for the treatment of lysosomal storage diseases, as they would be more potent (i.e. more inhibitory) in the neutral ER than they would be inside of the acidic lysosome and therefore bind Hex A more tightly in the ER than in the lysosome.

### Example 4

### Rescue of Mutant Hex A by KSH-10 and PYR

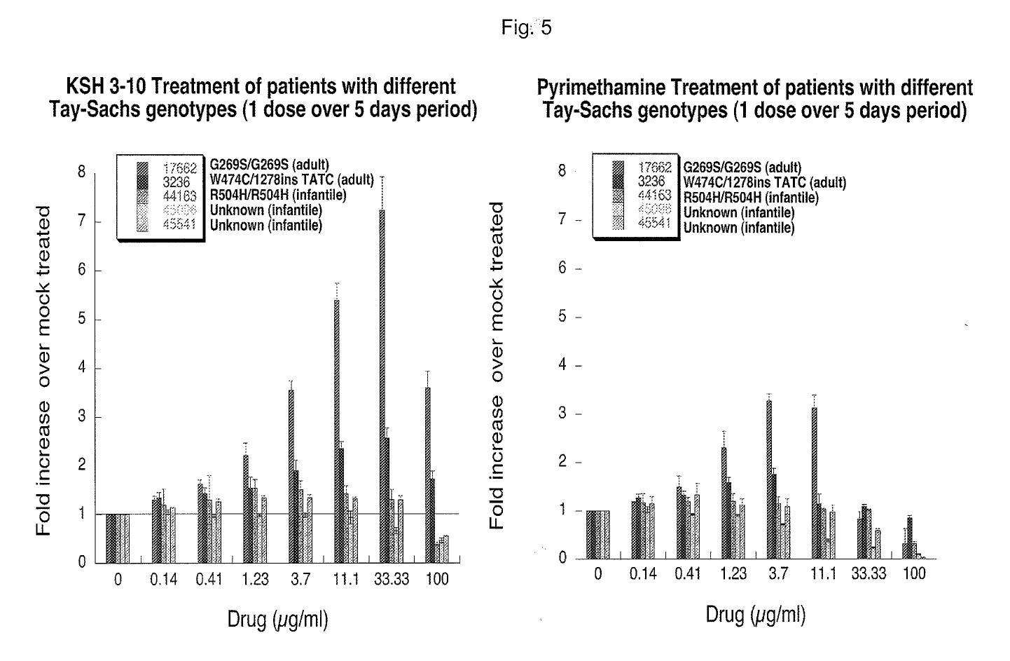

The abilities of KSH-10 and PYR to rescue mutant Hex A were compared in five different cultures of fibroblasts, each expressing a different mutation. The results are shown in FIG. 5. One culture of fibroblasts expressed the most common mutation, αG269S/G269S, associate with late onset GM2 gangliosidosis, adult Tay-Sachs disease (ATSD). Another culture had a rare W474C/Null genotype also associated with ATSD, and another had a rare R504H/R504H mutation associated with late infantile Tay-Sachs disease. Two of the other cultures were un-genotyped infantile Tay-Sachs disease fibroblast cell lines. Cells were grown in the indicated concentration of either PYR or KSH-10 for five days and were harvested and assayed for Hex A activity using a fluorescent artificial substrate, MUGS. The increase in Hex A activity relative to that of untreated cells is given, i.e., no change in activity=1. The decrease in Hex A activity at high concentrations represents cell toxicity.

Being a PYR derivative, KSH-10 is assumed to have a bio-availability that is similar to that of PYR. It also has the same ability to inhibit Hex better at neutral pH than at acidic pH, as was shown in Example 3. However, its IC50s are higher than PYR, i.e. 13.6 μg/mL at pH 6.5 and 40.5 μg/mL at pH 4.5. Despite this, KSH-10 produces a response as good or a better than PYR in all Tay-Sachs cell lines tested, including the most common ATSD mutation, G269S (FIG. 5). As can be seen from FIG. 5, KSH-10 exhibits an ability to rescue Hex A activity that is at least as good as or better than that of PYR, particularly at concentrations in which PYR caused cell toxicity. For example, at a concentration of 33.33 μg/ml KSH-10, there was a 7 fold increase in Hex A activity. At the same concentration of PYR, there was no increase in activity and cell death was occurring. This data indicates that KSH-10 is a much better pharmacological chaperone for Hex A than is PYR.

### Example 5

### Effect of PYR Derivatives on Hex A Activity in ISD Cells

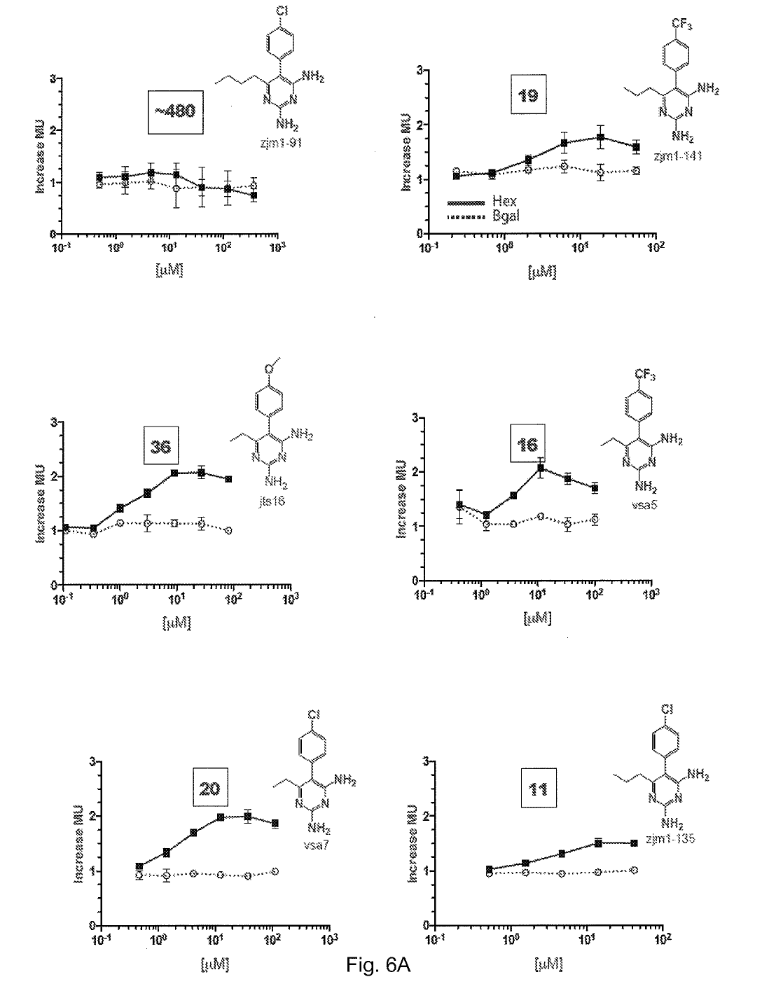

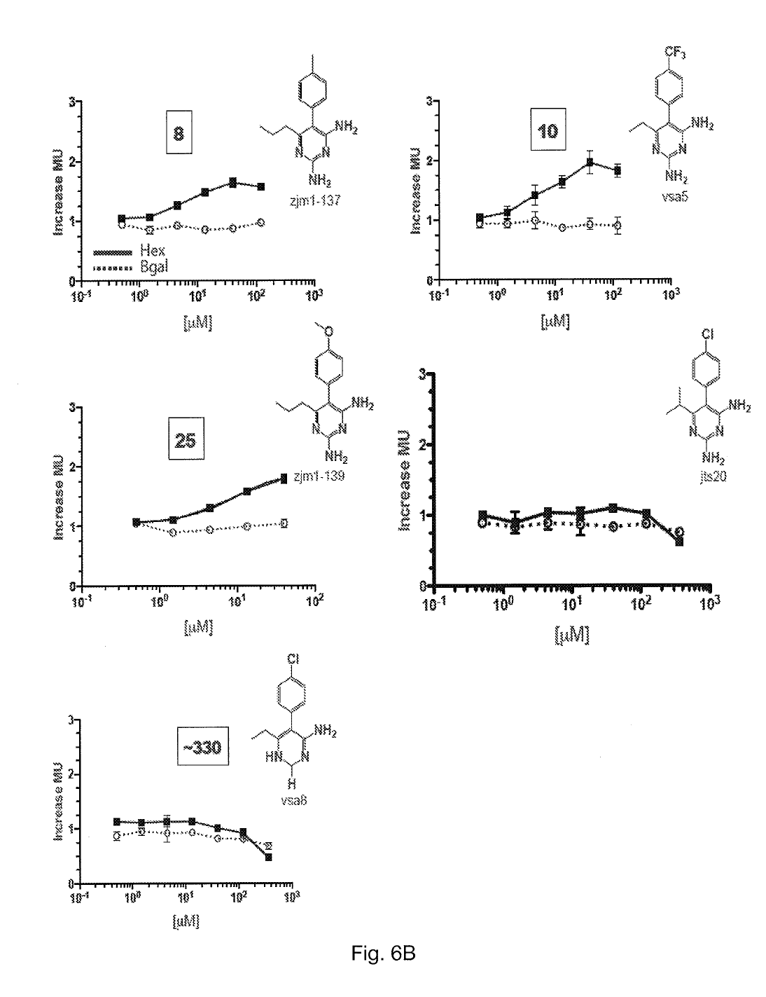

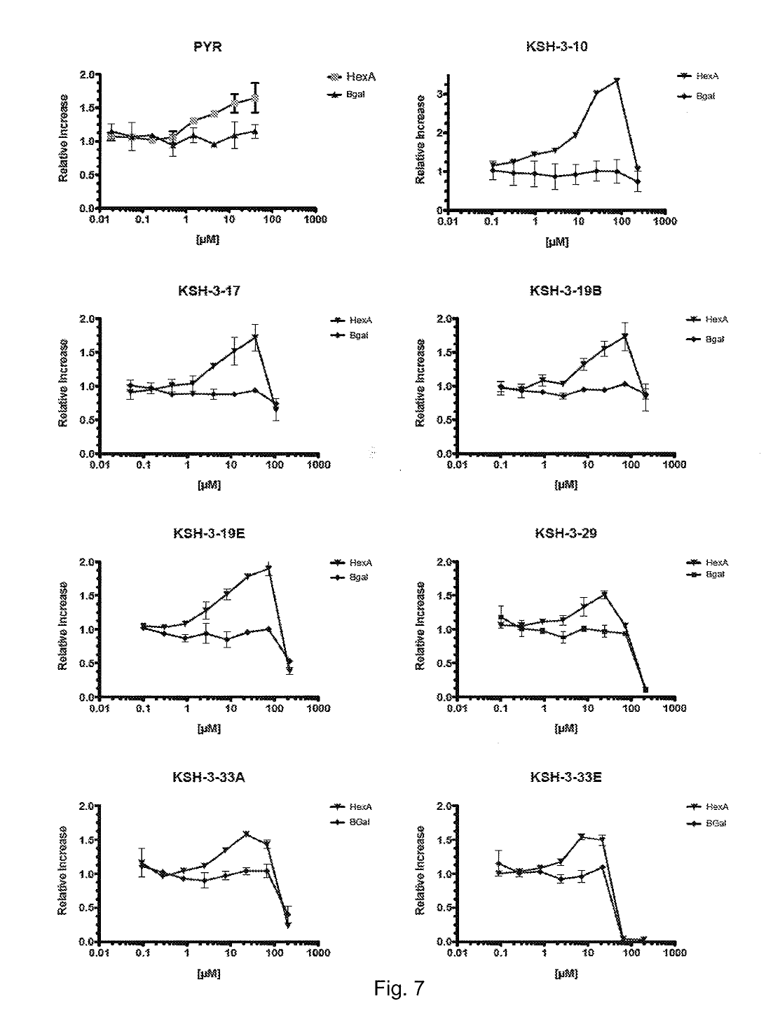

Experiments similar to those carried out in Example 4 were carried out using a variety of different PYR derivatives. These results are shown in FIGS. 6 and 7. As can be seen, each of the tested derivatives had the ability to rescue mutant Hex A activity. KSH-10 demonstrated an approximate 3 fold increase in its ability to rescue mutant Hex A activity at a concentration of 100 μM. In contrast, PYR only resulted in an approximate 1.5 fold increase in Hex A activity.

### Example 6

### Comparison of Hex A IC50 and EC50 Values

Using the graphs of FIGS. 5-7, the EC50 values for Hex A rescue were determined as well as the maximum increase and range of increase in Hex A activity. These values are tabulated in Table 2, together with the IC50 values for Hex A inhibition that were determined in Example 3.

TABLE 2
IC50 and EC50 values for PYR and PYR derivatives.

| | IC50 | IC50 | EC50 | Max | Range of |
|---|---|---|---|---|---|
| Name | (μM ± Err) | (μM) | (μM) | Increase | Increase |
| PYR (vsa7) | 11 ± 1 | 10.8 | 3.619 | 1.4 | 4 |
| vsa4 | (2114) | | | | |
| vsa5 | 15 ± 1 | 14.7 | 2.238 | 1.4 | 3 |
| vsa6 | (936 ± 4) | 936 | | | |
| vsa8 | (967 ± 1) | 967 | | | |
| vsa9 | (400) | | | | |
| zjm1-91 | (1363 ± 89) | 1363 | | | |
| zjm1-111 | (600 ± 1) | 600 | | | |
| zjm1-113 | (870 ± 5) | 870 | | | |
| zjm1-115 | 142 ± 3 | 142 | | | |
| zjm1-135 | 18 ± 1 | 17.9 | 7.138 | 1.4 | 3 |
| zjm1-137 | 28 ± 1 | 28.4 | 4.596 | 2.0 | 3 |
| zjm1-139 | 28 ± 1 | 28.2 | 14.22 | 2.3 | 4 |
| zjm1-141 | 16 ± 1 | 15.9 | 11.59 | 1.5 | 3 |
| jts14 | 23 ± 1 | 23.1 | 7.197 | 1.4 | 4 |
| jts16 | 18 ± 1 | 17.7 | 5.144 | 1.3 | 4 |
| jts20 | 406 ± 2 | 406 | | | |
| jts22 | 556 ± 1 | 556 | | | |
| ksh3-05 | (1232 ± 1) | 1232 | | | |
| ksh3-14a | (923 ± 2) | 923 | | | |
| ksh3-14b | (1722 ± 3) | 1722 | | | |
| ksh3-14c | (3826 ± 5) | 3826 | | | |
| ksh3-14d | (10361 ± 5) | | | | |
| ksh3-14e | (1227 ± 2) | 1227 | | | |
| ksh3-17 | 50 ± 1 | 50.4 | 8.236 | 1.8 | 4 |
| ksh3-19b | 72 ± 2 | 72.3 | 4.439 | 1.5 | 4 |
| ksh3-19c | 44 ± 1 | 43.7 | 4.947 | 1.9 | 4 |
| ksh3-19d | 34 ± 1 | 34.5 | 6.672 | 2.1 | 5 |
| ksh3-19e | 47 ± 1 | 47.4 | 16.36 | 2.2 | 3 |
| ksh3-29 | 37 ± 1 | 36.6 | 12.09 | 1.6 | 3 |
| ksk3-33a | 39 ± 1 | 39.1 | 12.54 | 1.4 | 4 |
| ksh3-33c | (825) | | | | |
| ksh3.33d | 50 ± 1 | 50.4 | 14.84 | 1.7 | 3 |
| ksh3.33e | 24 ± 1 | 24.4 | | 1.6 | 1 |
| zjm7-67 | 31 ± 1 | 30.8 | 6.457 | 1.7 | 2 |
| zjm7-69 | 55 ± 1 | 55 | 10.04 | 1.9 | 4 |
| ksh3-10 | 32 ± 1 | 32 | 38.45 | 2.6 | 4 |
| Pyr | | 14 | 8.113 | 2.0 | 4 |
| zjm7-27 | 176 ± 1 | 176 | | 1.4 | 1 |

*brackets indicate that the IC50 was extrapolated from an incomplete dose response curve

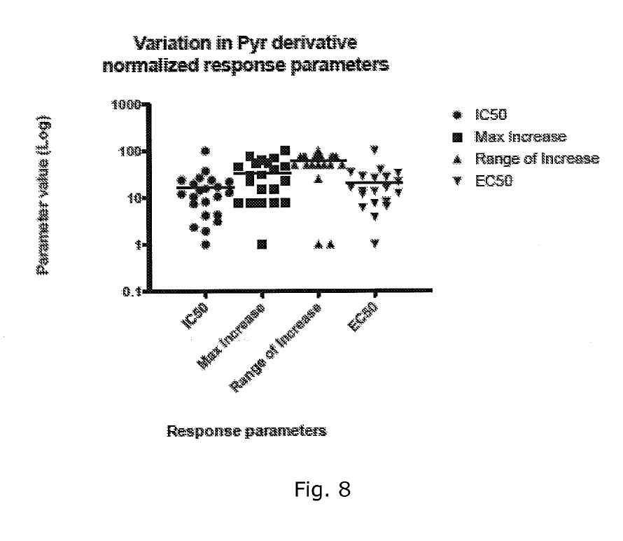

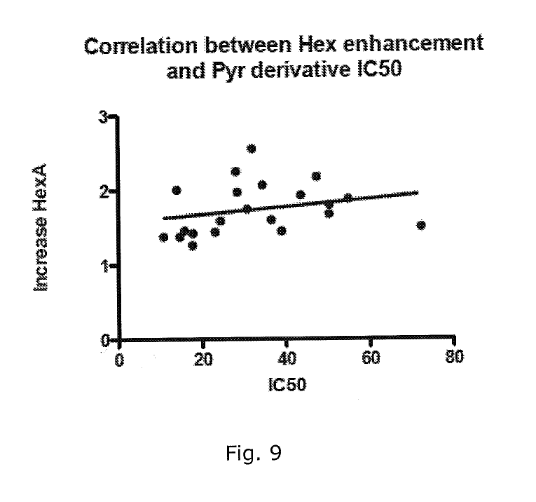

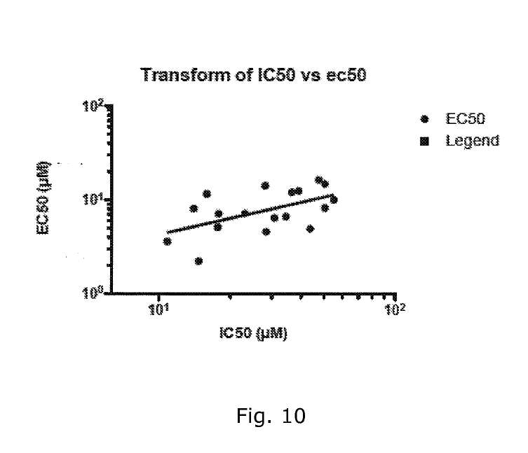

The data from Table 2 is shown in FIGS. 8, 9, and 10. FIG. 8 shows the variation in the normalized response parameters of the PYR derivatives. FIG. 9 shows the correlation between enhancement in Hex A activity and PYR derivative IC50. There is a slightly positive correlation, suggesting that the IC50 and Hex A enhancement potential may be related. FIG. 10 shows the correlation between the EC50 value and the IC50 value. The positive correlation here suggests that the EC50 and the IC50 values are related.

### Example 7

### Improved Enhancement of HexA Levels by PYR Derivatives (PYRdCl)

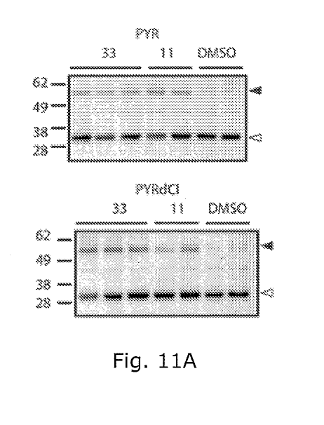

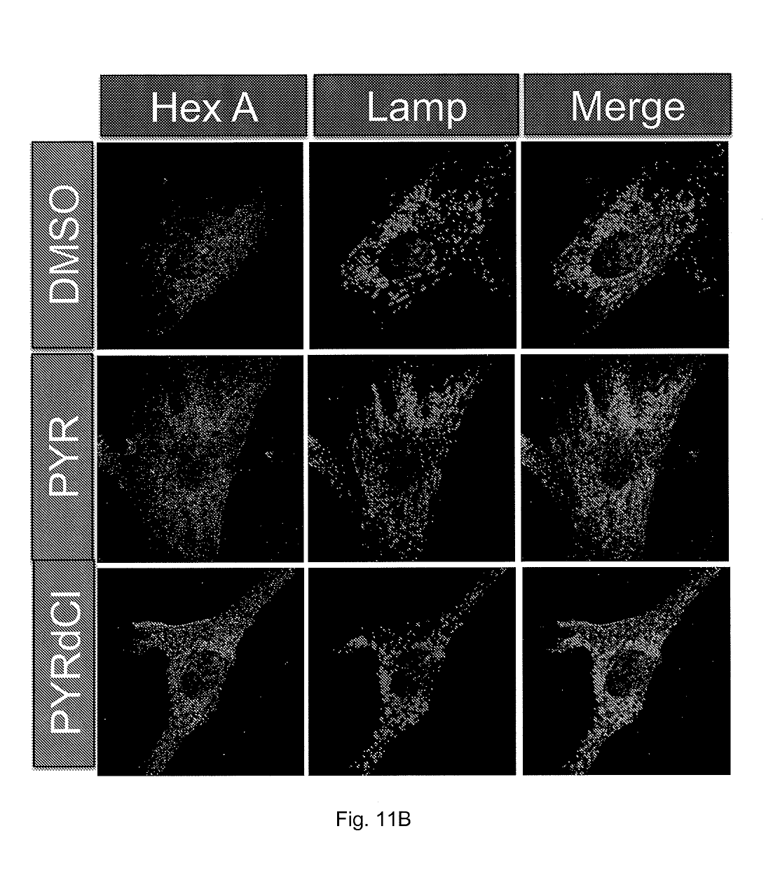

FIG. 11A shows increased levels of mature lysosomally derived Hex α-subunit (˜55 kDa) are seen in PYRdCl (KSH-10) relative to PYR treated patient cells. Lysates from patient Fibroblasts described above treated for 5 days with PYR (33 or 11 μg/mL) or PYRdCl (33 or 11 μg/mL) were resolved by SDS-PAGE, transferred to nitrocellulose, probed with a rabbit polyclonal antibody against HexA and followed by anti-Rabbit peroxidase conjugated Ab. Bands were visualized using chemiluminescent substrates. FIG. 11B shows levels and colocalization (yellow) of HexA (green) in lysosomes (LAMP, red) from p.G269S/p.G269S aHex patient fibroblasts following treatment with PYR or PYRdCl. Treated fibroblasts were permeabilized and probed with HexA rabbit antibody pre-absorbed with purified human HexB and Lamp1 mouse antibody. Primary Ab binding was visualized using the corresponding secondary anti-rabbit FITC (green) conjugated Ab or anti-mouse TRITC conjugated Ab (red). Colocalization of HexA and LAMP staining of the corresponding merged images is shown panels labeled MERGE.

### Example 8

### Increased Intracellular GM2 Hydrolysis in PYRdCl (KSH-10)

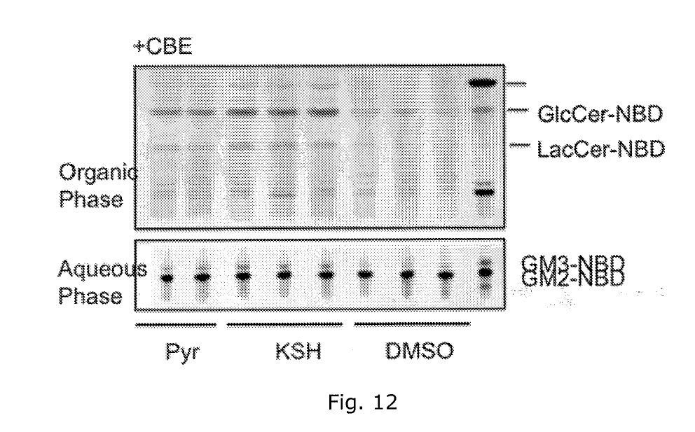

As shown in FIG. 12, patient cells treated with PYRdCl (11 μg/ml) resulted in greater intracellular hydrolysis of a fluorescent derivative of GM2 Ganglioside compared to cells treated with PYR (33 μg/ml). Patient fibroblasts (described above) were treated with either PYR or PYRdCl for 10 days. Prior to evaluation of the GM2 hydrolytic activity of lysosomal HexA, cells were treated with Conduritol b-epoxide (Glucocerebrosidase inhibitor) to limit hydrolysis beyond (GlcCer) loaded with a fluorescent derivative of GM2 ganglioside for 8 hrs. Neutral glycolipids and gangliosides were separated by Folch extraction of the treated cells. Gangliosides and glycolipids were resolved by High Performance Thin Layer Chromatography and visualized by Fluorescence Imaging (Storm Imager, Molecular Devices).

### Example 9

### Isozyme/Mutant Hex Activity PYR vs PYRdCl (KSH-10)

TABLE 3

pH 4.5

| | | | |
|---|---|---|---|
| pH 4.5 | HexA | HexA | pH 7 |

| | HexA | HexB | wt | G269S | HexA | HexB |
|---|---|---|---|---|---|---|
| Pyr | 8.9 | 9.1 | 14 | 8.5 | 3.1 | 4.5 |
| PyrdCl | 30 | 38 | 27 | 27 | 11 | 15 |

As shown in Table 3, differences in enzyme enhancement efficacy of PYRdCl versus PYR can not be attributed to differences in their inhibitory activity (IC₅₀) towards isozymes HexA and HexB or normal HexA and mutant HexA. The IC₅₀ values of PYR and PYRdCl against purified HexA and HexB were similar. At neutral pH, IC₅₀ values of PYR and PYRdCl was reduced approximately three-fold for both isozymes. The IC50 values of PYR and PYRdCl at pH 4.5 for the HexA isozyme enriched from normal or Adult TaySach fibroblasts bearing the p.G269S were similarly affected. Experiments were performed using 1.6 mM 4-methylumbelliferone-b-N-Acetylglucosamine substrate.

### Example 10

### Viability of PYR vs PYRdCl Treated Cells

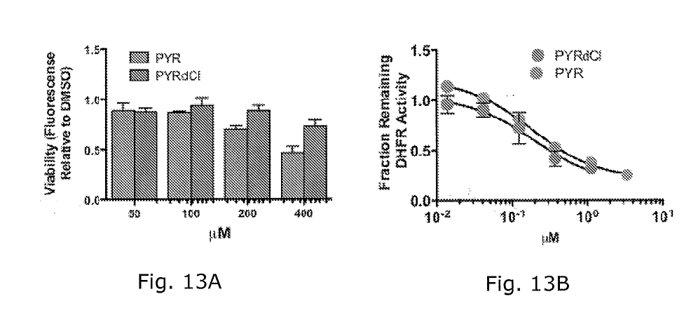

In FIG. 13A, compared to PYRdCl treated patient fibroblasts the viability of PYR treated cells at concentrations greater than 100 μM is reduced by more than 20%. The decreased HexA activity enhancement by PYR relative to PYRdCl at higher concentrations can be attributed to the decreased viability of cells at these levels of compound. In FIG. 13B, the inhibitory activity of both compounds against purified recombinant human DHFR are similar (IC₅₀=0.2 μM), implying that differential toxicity of the compounds can not be attributed solely to their effects on DHFR. Viability of cells following five days of treatment with PYR and PYRdCl was determined using Alamar Blue (a cell-permeable non-fluorescent indicator that upon reduction in metabolically active cells becomes fluorescent).

The description as set forth is not intended to be exhaustive or to limit the scope of the invention. Many modifications and variations are possible in light of the above teaching without departing from the spirit and scope of the following claims. It is contemplated that the use of the present invention can involve components having different characteristics. It is intended that the scope of the present invention be defined by the claims appended hereto, giving full cognizance to equivalents in all respects.

1. A method of treating a lysosomal storage disease, the method comprising administering a pyrimethamine derivative to a subject in need thereof.

2. The method of claim 1, wherein the lysosomal storage disease is selected from the group consisting of GM1 gangliosidosis, GM2 gangliosidosis, Fabry disease, Gaucher disease, Sanfilippo syndrome, and Morquio disease.

3. The method of claim 2, wherein the GM2 gangliosidosis is selected from Tay-Sachs disease, Sandhoff disease, and AB variant.

4. The method of claim 3, wherein the lysosomal storage disease is Tay-Sachs disease.

5. The method of claim 1, wherein the pyrimethamine derivative is of general formula I: 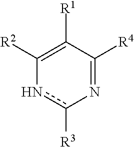
R¹ is a substituted aryl, unsubstituted aryl, substituted heteroaryl, or unsubstituted heteroaryl;
R² is H, NH₂, or alkylamino;
R³ is H, NH₂, ═O, or alkylamino, and when R³ is H, NH₂, or alkylamino, [symbol] is a double bond, when R³ is ═O, [symbol] is a double single bond; and
R⁴ is a substituted or unsubstituted hydrocarbyl,
wherein when R¹ is 4-chlorophenyl and when [symbol] is a double bond, R² is other than NH₂, R³ is other than NH₂, and R⁴ is other than ethyl.

6. The method of claim 5, wherein R¹ is a substituted aryl or unsubstituted heteroaryl; R² is H, NH₂; R³ is NH₂or alkylamino and [symbol] is a double bond, and R⁴ is a substituted or unsubstituted alkyl.

7. The method of claim 5, wherein R¹ is a substituted phenyl, substituted thiophene or unsubstituted thiophene; R² is H, NH₂; R³ is NH₂ or alkylamino and [symbol] is a double bond, and R⁴ is a substituted or unsubstituted C₁-C₁₀ alkyl.

8. The method of claim 7, wherein R⁴ is a C₁-C₄ alkyl.

9. The method of claim 8, wherein R¹ is: 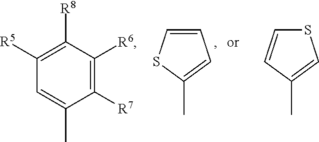
R⁵, R⁶, and R⁷ is independently H, substituted or unsubstituted hydrocarbyl; R⁸ is H, substituted haloalkyl, unsubstituted haloalkyl, halo, substituted hydrocarbyl, unsubstituted hydrocarbyl, substituted alkoxy, or unsubstituted alkoxy.

10. The method of claim 9, wherein R⁵, R⁶, and R⁷ are each independently H or CH₃; and R⁸ is CF₃, CH₃, —O—CH₃, F, H, or Cl.

11. The method of claim 1, wherein the pyrimethamine derivative is selected from the group consisting of:

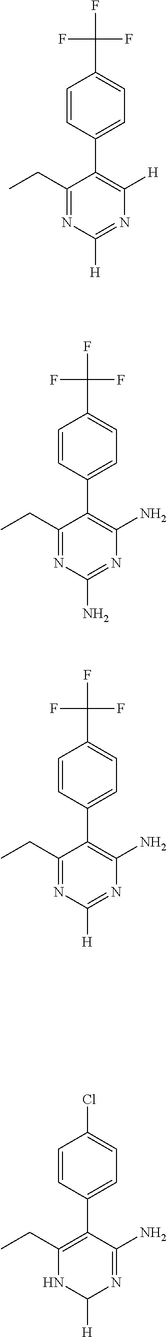 [SMILES: CCc1ncncc1-c1ccc(C(F)(F)F)cc1 ; CCc1nc(N)nc(N)c1-c1ccc(C(F)(F)F)cc1 ; CCc1ncnc(N)c1-c1ccc(C(F)(F)F)cc1 ; CCC1=C(c2ccc(Cl)cc2)C(N)=NCN1]

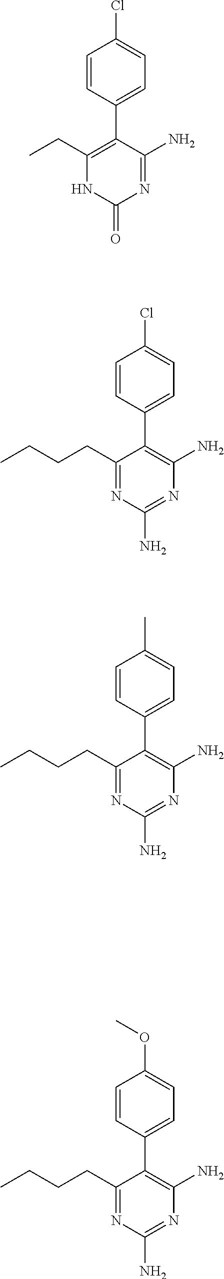 [SMILES: CCc1[nH]c(=O)nc(N)c1-c1ccc(Cl)cc1 ; CCCCc1nc(N)nc(N)c1-c1ccc(Cl)cc1 ; CCCCc1nc(N)nc(N)c1-c1ccc(C)cc1 ; CCCCc1nc(N)nc(N)c1-c1ccc(OC)cc1]

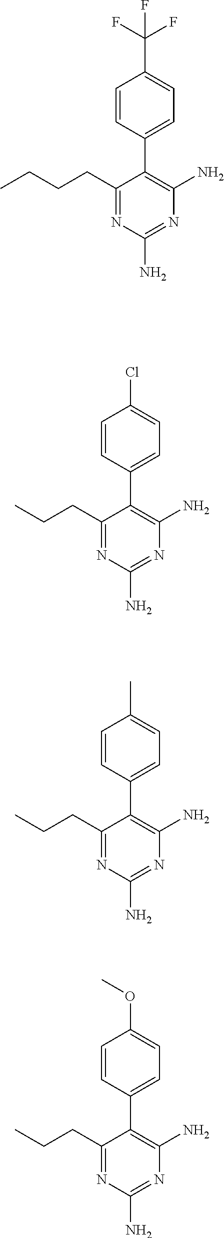 [SMILES: CCCCc1nc(N)nc(N)c1-c1ccc(C(F)(F)F)cc1 ; CCCc1nc(N)nc(N)c1-c1ccc(Cl)cc1 ; CCCc1nc(N)nc(N)c1-c1ccc(C)cc1 ; CCCc1nc(N)nc(N)c1-c1ccc(OC)cc1]

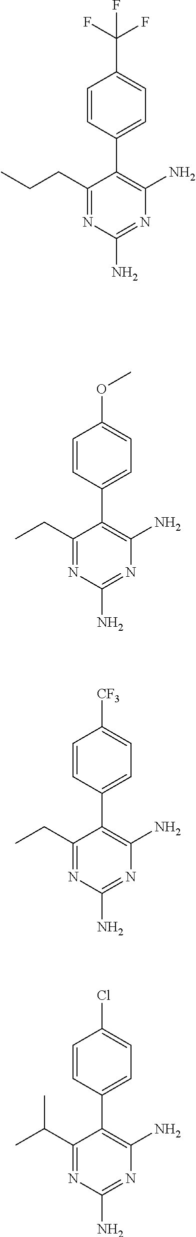 [SMILES: CCCc1nc(N)nc(N)c1-c1ccc(C(F)(F)F)cc1 ; CCc1nc(N)nc(N)c1-c1ccc(OC)cc1 ; CCc1nc(N)nc(N)c1-c1ccc(C(F)(F)F)cc1 ; CC(C)c1nc(N)nc(N)c1-c1ccc(Cl)cc1]

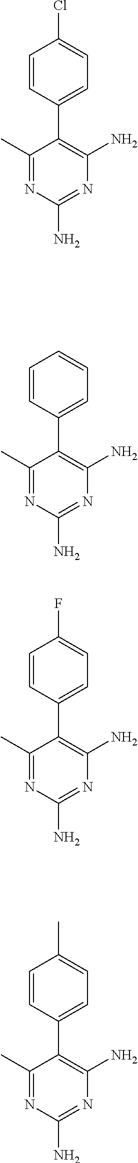 [SMILES: Cc1nc(N)nc(N)c1-c1ccc(Cl)cc1 ; Cc1nc(N)nc(N)c1-c1ccccc1 ; Cc1nc(N)nc(N)c1-c1ccc(F)cc1 ; Cc1ccc(-c2c(C)nc(N)nc2N)cc1]

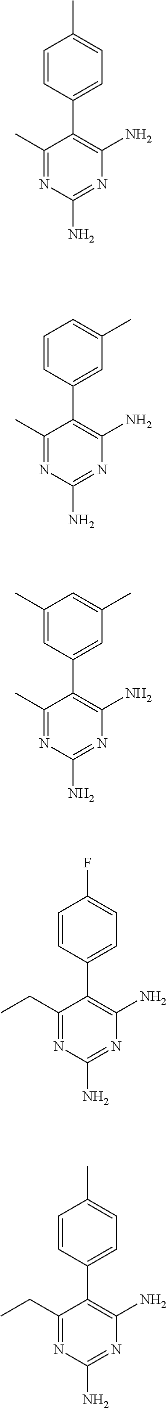

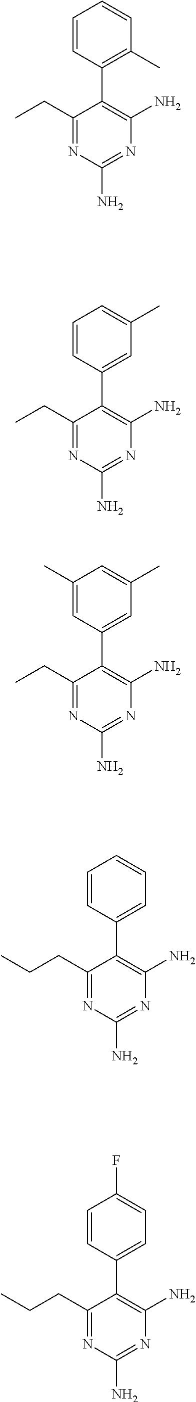

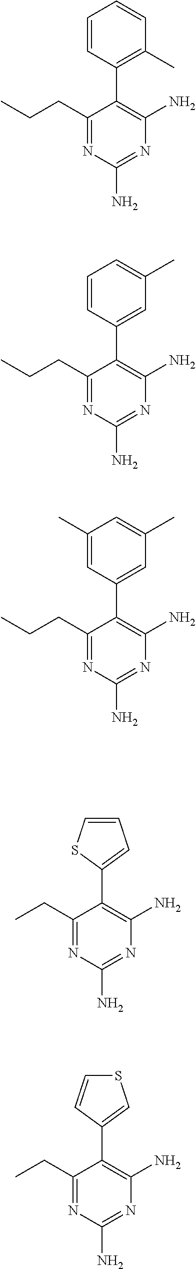

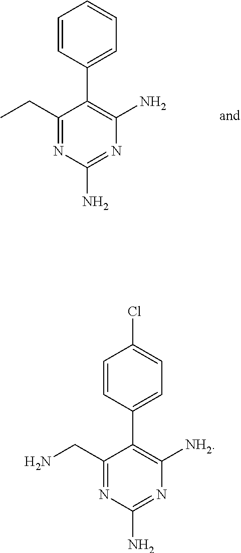 [SMILES: CCc1nc(N)nc(N)c1-c1ccccc1 ; NCc1nc(N)nc(N)c1-c1ccc(Cl)cc1]

12. The method of claim 11, wherein the pyrimethamine derivative is

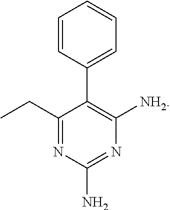 [SMILES: CCc1nc(N)nc(N)c1-c1ccccc1]

13-25. (canceled)

26. The method of claim 1, wherein the lysosomal storage disease is selected from the group consisting of mucopolysaccharidosis diseases; Tay-Sachs disease; Sandhoff disease; GM1 gangliosidosis; Fabry disease; metachromatic leukodystrophy; Pompe disease; fucosidosis; alpha-mannosidosis; beta-mannosidosis; ceroid lipofuscinosis; Gaucher disease; Hermansky-Pudlak syndrome; Amaurotic idiocy; Tangier disease; aspartylglucosaminuria; congenital disorder of glycosylation, type Ia; Chediak-Higashi syndrome; macular dystrophy, corneal, 1; cystinosis, nephropathic; Fanconi-Bickel syndrome; Farber lipogranulomatosis; fibromatosis; geleophysic dysplasia; a glycogen storage disease; immunoosseous dysplasia, Schimke type; lipidosis; lipase b; a mucolipidosis; neuraminidase deficiency with beta-galactosidase deficiency; Niemann-Pick disease; Refsum disease; Sea-blue histiocyte disease; infantile sialic acid storage disorder; sialuria; multiple sulfatase deficiency; triglyceride storage disease with impaired long-chain fatty acid oxidation; Winchester disease; Wolman disease; Deoxyribonuclease I-like 1 disorder, arylsulfatase E disorder; ATPase, H+ transporting, lysosomal, subunit 1 disorder; Ras-associated protein rab9 disorder; chondrodysplasia punctata 1, X-linked recessive disorder; lysosome-associated membrane protein 2 disorder; Menkes syndrome; congenital disorder of glycosylation, type Ic; and sialuria.

27. The method of claim 1, wherein:
R¹ is a substituted aryl or unsubstituted heteroaryl;
R² is H or NH₂;
R³ is NH₂ or alkylamino; [symbol]
is a double bond; and
R⁴ is substituted or unsubstituted alkyl,
wherein when R¹ is phenyl, 4-fluorophenyl, 4-trifluoromethyl-phenyl, or 4-chlorophenyl, R² is other than NH₂, R³ is other than NH₂, and/or R⁴ is other than ethyl, and
wherein when R¹ is phenyl substituted with F or CF₃, R¹ is 4-fluorophenyl or 4-trifluoromethyl-phenyl.

28. The method of claim 1, wherein:
R¹ is a substituted phenyl, substituted thiophene or unsubstituted thiophene;
R² is H, NH₂;
R³ is NH₂ or alkylamino, [symbol]
is a double bond; and
R⁴ is a substituted or unsubstituted C₁-C₁₀ alkyl,
wherein when R¹ is 4-fluorophenyl, 4-trifluoromethyl-phenyl, or 4-chlorophenyl, R² is other than NH₂, R³ is other than NH₂, and/or R⁴ is other than ethyl, and
wherein when R¹ is phenyl substituted with F or CF₃, R¹ is 4-fluorophenyl or 4-trifluoromethyl-phenyl.

29. The method of claim 28, wherein R⁴ is a C₁-C₄ alkyl.

30. The method of claim 28, wherein R¹ is: 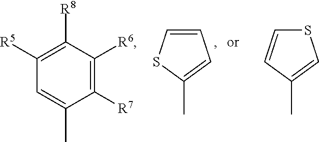
wherein R⁵, R⁶, and R⁷ is independently H, substituted or unsubstituted hydrocarbyl; is H, substituted haloalkyl, unsubstituted haloalkyl, halo, substituted hydrocarbyl, unsubstituted hydrocarbyl, substituted alkoxy, or unsubstituted alkoxy.

31. The method of claim 30, wherein R⁵, R⁶, and R⁷ are each independently H or CH₃; and R⁸ is CF₃, CH₃, —O—CH₃, F, H, or Cl.

32. The method of claim 1, wherein:
R¹ is a substituted aryl, unsubstituted aryl, substituted heteroaryl, or unsubstituted heteroaryl;
R² is H, NH₂, or alkylamino;
R³ is H, NH₂, ═O, or alkylamino, and when R³ is H, NH₂, or alkylamino, [symbol] is a double bond, when R³ is ═O, [symbol] is a single bond; and
R⁴ is an unsubstituted hydrocarbyl or a hydrocarbyl substituted with cycloalkyl, heterocyclyl, hydroxyalkyl, benzyl, carbonyl, halo, haloalkyl, perfluoroalkyl, perfluoroalkoxy, alkyl, alkenyl, alkynyl, hydroxy, oxo, mercapto, alkylthio, aryl, heteroaryl, aryloxy, heteroaryloxy, aralkyl, heteroaralkyl, aralkoxy, heteroaralkoxy, HO—(C═O)—, amido, amino, alkylamino, dialkylamino, cyano, nitro, carbamoyl, alkylcarbonyl, alkoxycarbonyl, alkylaminocarbonyl, dialkylaminocarbonyl, arylcarbonyl, aryloxycarbonyl, alkylsulfonyl, or arylsulfonyl,
wherein when R¹ is phenyl, 4-fluorophenyl, 4-trifluoromethyl-phenyl, or 4-chlorophenyl and when [symbol] is a double bond, R² is other than NH₂, R³ is other than NH₂, and/or R⁴ is other than ethyl,
wherein when R¹ is phenyl substituted with F or CF₃, R¹ is 4-fluorophenyl or 4-trifluoromethyl-phenyl, and
wherein the IC50 value for HexA inhibition of the derivative is less than about 100 μM.

33. The method of claim 1, wherein:
R¹ is substituted aryl,
R² and R³ are independently H or NH₂; and
R⁴ is (C₁-C₄) alkyl,
wherein when R¹ is phenyl, 4-fluorophenyl, 4-trifluoromethyl-phenyl, or 4-chlorophenyl and when [symbol] is a double bond, R² is other than NH₂, R³ is other than NH₂, and/or R⁴ is other than ethyl, and
wherein when R¹ is phenyl substituted with F or CF₃, R¹ is 4-fluorophenyl or 4-trifluoromethyl-phenyl.
